(19) United States
(12) Patent Application Publication
(10) Pub. No.: US 2015/0037371 A1
(43) Pub. Date: Feb. 5, 2015

(54) COMPOSITIONS, METHODS, AND KITS FOR ENHANCING THE IMMUNOGENICITY OF PATHOGENIC ANTIGENS

(71) Applicant: The Administrators of the Tulane Educational Fund, New Orleans, LA (US)

(72) Inventor: Samuel J. LANDRY, New Orleans, LA (US)

(21) Appl. No.: 14/514,095

(22) Filed: Oct. 14, 2014

Related U.S. Application Data
(63) Continuation of application No. 13/123,092, filed on Apr. 22, 2011, now abandoned.
(60) Provisional application No. 61/103,793, filed on Oct. 8, 2008.

(51) Int. Cl. C07K 14/005; A61K 39/21; G01N 33/569; C12N 7/00

(52) U.S. Cl. CPC C07K 14/005; C12N 7/00; A61K 39/21; G01N 33/56988; C12N 2740/16122; C12N 2740/16134; G01N 2500/04; G01N 2333/162
USPC 424/208.1; 530/403; 530/395; 435/320.1; 435/69.3; 436/501; 506/9

(57) ABSTRACT

The invention provides a method of enhancing the immunogenicity of pathogenic antigens by removing or disrupting intrachain disulfide bonds responsible for maintaining tertiary protein structure. Removal of one or more disulfide bonds can increase the titer of neutralizing antibodies to a pathogen (e.g., a bacterium, fungus, virus, or parasite). The invention also features vaccines, expression vectors, and methods for the manufacture and use thereof.

### STATEMENT OF FEDERALLY FUNDED RESEARCH

This research has been sponsored in part by NIH grant number R21-AI42702. The government has certain rights to the invention.

### FIELD OF THE INVENTION

The invention provides a method of enhancing the immunogenicity of pathogenic antigens by removing or disrupting intrachain disulfide bonds normally involved in the maintenance of tertiary protein structure. Removal of one or more disulfide bonds can increase the titer of neutralizing antibodies to a pathogen (e.g., a bacterium, fungus, virus, or parasite). The invention also features vaccines, expression vectors, and methods for the manufacture and use thereof.

### BACKGROUND OF THE INVENTION

The specificity of CD4+ T cell responses could be crucial to vaccine effectiveness. The prevailing view among AIDS vaccine researchers is that a highly effective vaccine will elicit neutralizing antibody and cytotoxic T cells (CTLs). Both arms of the immune response require CD4+ helper T cells. Mutation of a CD4+ epitope was shown to be a viable mechanism for immune escape by lymphocytic choriomeningitis virus (LCMV) in mice (Ciurea et al., Nat. Med. 7:795 (2001)), and CD4+ escape mutants have been described in chronic hepatitis C (HCV) infections (Wang et al., J. Immunol. 162:4177 (1999); Wang et al., J. Mol. Evol. 54:465 (2002); and Puig et al., Hepatology 44:736 (2006)). Some human immunodeficiency virus (HIV) mutations whose selection cannot be explained by escape from CD8+ T cells are located in CD4+ epitopes (Allen et al., J. Virol. 79:13239 (2005)). Responses to subdominant CD4+ epitopes could be especially important for protection when escape mutations arise in the dominant epitopes (Zhou et al., Vaccine 24:2451 (2006)). CD4+ epitopes appear to be less conserved than CD8+ epitopes, possibly just because CD4+ epitopes are longer (Smith et al., AIDS Res. Hum. Retroviruses 21:14 (2005)). Since escape by mutation of CD4+ epitopes may limit vaccine effectiveness, increasing the breadth of CD4+ responses could be an important goal of vaccine design.

Vaccine design is hampered by a poor understanding of CD4+ T cell priming as well as of the mechanics of T cell help. Although the basic importance of the CD4+ helper T cell response is well recognized (Rosenberg et al., Science 278:1447 (1997) and Letvin, J. Clin. Invest. 110:15 (2002)), crucial aspects of the response remain largely unexplored, including the factors that determine epitope immunodominance in the CD4+ response and the relationship of specificity in the CD4+ T cell response to specificity in the antibody response. The definition of these factors and relationships will allow for the production of vaccines with improved immunogenicity for both therapeutic and prophylactic administration. The need for improved vaccines is particularly urgent for the treatment of pathogens, such as HIV.

### SUMMARY OF THE INVENTION

In a first aspect, the invention features methods for improving the immunogenicity of a polypeptide antigen by removing or disrupting at least one intrachain disulfide bond of the antigen.

In a second aspect, the invention features a vaccine having a polypeptide antigen having at least one removed or disrupted intrachain disulfide bond.

In a third aspect, the invention features an expression vector having a nucleic acid that encodes a polypeptide antigen having at least one removed or disrupted intrachain disulfide bond.

In a fourth aspect, the invention features a method of treating a mammal infected with or at risk of becoming infected with a pathogen by administering a polypeptide antigen having at least one removed or disrupted intrachain disulfide bond.

In a fifth aspect, the invention features a method of treating a mammal infected with or at risk of becoming infected with a pathogen by administering a polypeptide antigen having at least one removed or disrupted intrachain disulfide bond.

In a sixth aspect, the invention features a method of treating a mammal infected with or at risk of becoming infected with a pathogen by administering an expression vector having a nucleic acid that encodes a polypeptide antigen having at least one removed or disrupted intrachain disulfide bond.

In a seventh aspect, the invention features a method of manufacturing a vaccine agent with enhanced immunogenicity in a mammal by synthesizing a polypeptide antigen having at least one removed or disrupted intrachain disulfide bond.

In an eighth aspect, the invention features a method of manufacturing a vaccine agent with enhanced immunogenicity in a mammal by contacting an expression vector having a nucleic acid that encodes a polypeptide antigen having at least one removed or disrupted intrachain disulfide bond with a cell followed by isolating the polypeptide agent.

In a ninth aspect, the invention features a method of identifying an antibody that specifically binds a pathogenic antigen by contacting an antibody with a viral, fungal, bacterial, or parasitic antigen in which at least one disulfide bond has been removed or disrupted and then identifying an antibody that binds the antigen with a dissociation constant of less than 10⁻⁶M.

In a tenth aspect, the invention features a kit containing a vaccine having a polypeptide antigen having at least one removed or disrupted intrachain disulfide bond. of the invention, a pharmaceutically acceptable carrier, excipient, or diluent, and instructions for use thereof. In one embodiment, the kit contains an expression vector encoding the nucleic acid of a polypeptide antigen having at least one removed or disrupted intrachain disulfide bond. In another embodiment, the kit includes an adjuvant.

In one embodiment of any aspect of the invention, the polypeptide antigen can elicit a neutralizing antibody when administered to a mammal, such as a human. In another embodiment, the antigen is not completely denatured or is glycosylated. In a further embodiment, one or more cysteine residues in the antigen are substituted (e.g., with alanine) or deleted in order to remove or disrupt at least one disulfide bond. A disulfide bond can be removed or disrupted by exposure to a reducing agent.

In another embodiment of any aspect of the invention, the polypeptide antigen is a viral, fungal, bacterial, or parasitic antigen. In one embodiment, a viral, fungal, bacterial, or parasitic antigen has a neutralizable epitope. In another embodiment, the polypeptide antigen is a human immunodeficiency virus (HIV) envelope glycoprotein, or fragment thereof, including gp120, gp41, and gp160. The HIV envelope glycoprotein can contain an intrachain disulfide bond that forms a variable (V) loop region, such as the V3 and V4 loops. In a further embodiment, the polypeptide antigen is not derived from the hepatitis C virus (HCV).

### DEFINITIONS

The term “antibody” as used interchangeably herein, includes whole antibodies or immunoglobulins and any antigen-binding fragment or single chains thereof. Antibodies, as used herein, can be mammalian (e.g., human or mouse), humanized, chimeric, recombinant, synthetically produced, or naturally isolated. In most mammals, including humans, antibodies have at least two heavy (H) chains and two light (L) chains connected by disulfide bonds. Each heavy chain consists of a heavy chain variable region (abbreviated herein as V_{H}) and a heavy chain constant region. The heavy chain constant region consists of three domains, C_{H}1, C_{H}2, and C_{H}3 and a hinge region between C_{H}1 and C_{H}2. Each light chain consists of a light chain variable region (abbreviated herein as V_{L}) and a light chain constant region. The light chain constant region consists of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. Antibodies of the present invention include all known forms of antibodies and other protein scaffolds with antibody-like properties. For example, the antibody can be a human antibody, a humanized antibody, a bispecific antibody, a chimeric antibody, or a protein scaffold with antibody-like properties, such as fibronectin or ankyrin repeats. The antibody also can be a Fab, Fab′2, scFv, SMIP, diabody, nanobody, aptamers, or a domain antibody. The antibody can have any of the following isotypes: IgG (e.g., IgG1, IgG2, IgG3, and IgG4), IgM, IgA (e.g., IgA 1, IgA2, and IgAsec), IgD, or IgE.

The term “antibody fragment”, as used herein, refers to one or more fragments of an antibody that retain the ability to specifically bind to an antigen. The antigen-binding function of an antibody can be performed by fragments of a full-length antibody. Examples of binding fragments encompassed within the term “antigen-binding portion” of an antibody include but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544 (1989)), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., Science 242:423 (1988) and Huston et al., Proc. Natl. Acad. Sci. USA 85:5879 (1988), each herein incorporated by reference). These antibody fragments are obtained using conventional techniques known to those with skill in the art, and the fragments are screened for utility in the same manner as are intact antibodies. Antibody fragments can be produced by recombinant DNA techniques, or by enzymatic or chemical cleavage of intact immunoglobulins.

The terms “effective amount” or “amount effective to” or “therapeutically effective amount” means an amount sufficient to produce a desired result, for example, the reduction of a viremia in a patient (e.g., a human) infected with a virus, or the prevention of a pathogenic infection in a patient at risk of infection.

By “expression vector” is meant a DNA construct that contains a promoter operably linked to a downstream gene or coding region (e.g., a cDNA or genomic DNA fragment, which encodes a polypeptide or polypeptide fragment). Introduction of the expression vector into a recipient cell (e.g., a prokaryotic or eukaryotic cell, e.g., a bacterium, yeast, insect cell, or mammalian cell, depending upon the promoter within the expression vector) allows the cell to express mRNA encoded by the expression vector, which is then translated into the encoded polypeptide or polypeptide fragment. Vectors for in vitro transcription/translation are also well-known in the art. An expression vector may be a genetically engineered plasmid, virus, or artificial chromosome derived from, e.g., a bacteriophage, adenovirus, retrovirus, poxvirus, or herpesvirus.

The term “human antibody,” as used herein, is intended to include antibodies, or fragments thereof, having variable regions in which both the framework and CDR regions are derived from human germline immunoglobulin sequences as described, for example, by Kabat et al., (Sequences of proteins of Immunological Interest, Fifth Edition, U.S. Department of Health and Human Services, NIH Publication No. 91-3242 (1991)). Furthermore, if the antibody contains a constant region, the constant region also is derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences (i.e., a humanized antibody or antibody fragment).

The term “humanized antibody” refers to any antibody or antibody fragment that includes at least one immunoglobulin domain having a variable region that includes a variable framework region substantially derived from a human immunoglobulin or antibody and complementarity determining regions (e.g., at least one CDR) substantially derived from a non-human immunoglobulin or antibody.

By an “improved immunogenicity” or “improving immunogenicity” in reference to an altered polypeptide antigen is meant an increase in the antibody titer in a test animal immunized with the altered polypeptide antigen relative to the antibody titer in a control test animal immunized with the same amount of an unaltered polypeptide antigen and/or an increase in the breath of binding specificity to epitopes present on the antigen of antibodies raised against the altered polypeptide antigen relative to antibodies raised against the unaltered polypeptide antigen under the same conditions. An improved immunogenicity can be a two-, three- four-, five-, six-, seven-, eight-, nine-, ten-, one hundred-, one thousand-, or even ten thousand-fold increase in the antibody titer relative to that obtained in the test animal with the unaltered polypeptide antigen. The polypeptide antigen may be altered, for example, by removing or disrupting at least one intrachain disulfide bond (e.g., by altering the coding sequence to remove one or more cysteine residues). Antibody titers and breadth of binding specificity may be measured using standard techniques in the art, such as an Enzyme-Linked Immunosorbent Assay (ELISA).

By “neutralizing antibody” is meant an antibody which is capable of specifically binding to a neutralizable antigen (e.g., a neutralizable epitope, or other antigenic determinant) of a pathogen (e.g., a bacterium, fungus, virus, or parasite) and substantially inhibiting or eliminating the biologically activity of the pathogen. Typically, a neutralizing antibody will inhibit the biologically activity of the pathogen by at least by about 50%, and preferably by 80%, 90%, 95%, 99% or more.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different antigenic determinants (epitopes), each monoclonal antibody is directed against a single determinant on the antigen. Monoclonal antibodies can be prepared using any art recognized technique and those described herein such as, for example, a hybridoma method, as described by Kohler et al., Nature 256:495 (1975), a transgcnic animal (e.g., Lonberg et al., Nature 368(6474):856 (1994)), recombinant DNA methods (e.g., U.S. Pat. No. 4,816,567, herein incorporated by reference), or using phage, yeast, or synthetic scaffold antibody libraries using the techniques described in, e.g., Clackson et al., Nature 352:624 (1991) and Marks et al., J. Mol. Biol. 222:581 (1991).

By “pharmaceutically acceptable carrier” is meant a carrier which is physiologically acceptable to the treated mammal while retaining the therapeutic properties of the compound with which it is administered. One exemplary pharmaceutically acceptable carrier is physiological saline. Other physiologically acceptable carriers and their formulations are known to one skilled in the art and described, e.g., in Remington's Pharmaceutical Sciences (18^{th} edition, ed. A. Gennaro, 1990, Mack Publishing Company, Easton, Pa.), incorporated herein by reference.

By “specifically bind” is meant the preferential association of an antibody or antibody fragment moiety to a target molecule (e.g., an antigen, such as a peptide, polypeptide, glycoprotein, or any other moiety with one or more antigenic determinants) or to a cell or tissue bearing the target molecule (e.g., a cell surface antigen, such as a receptor or ligand) and not to cells or tissues lacking the target molecule. It is recognized that a certain degree of non-specific interaction may occur between a binding moiety and a non-targeted molecule (present alone or in combination with a cell or tissue). Nevertheless, specific binding may be distinguished as mediated through specific recognition of the target antigen. Specific binding results in a much stronger association between the binding moiety (e.g., an antibody) and e.g., cells bearing the target molecule (e.g., an antigen) than between the binding moiety and e.g., cells lacking the target molecule. Specific binding typically results in greater than 2-fold, preferably greater than 5-fold, more preferably greater than 10-fold and most preferably greater than 100-fold increase in amount of bound binding moiety (per unit time) to e.g., a cell or tissue bearing the target molecule or marker as compared to a cell or tissue lacking that target molecule or marker. Binding moieties bind to the target molecule or marker with a dissociation constant of e.g., less than 10⁻⁶M, more preferably less than 10⁻⁷M, 10⁻⁸M, 10⁻⁹M, 10⁻¹⁰M, 10⁻¹¹M, or 10⁻¹²M, and most preferably less than 10⁻¹³M, 10⁻¹⁴M, or 10⁻¹⁵M. Specific binding to a protein under such conditions requires a binding moiety that is selected for its specificity for that particular protein. A variety of assay formats are appropriate for selecting binding moieties (e.g., antibodies) capable of specifically binding to a particular target molecule. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

The term “substantial identity” or “substantially identical,” when used in the context of comparing a polynucleotide or polypeptide sequence to a reference sequence, means that the polynucleotide or polypeptide sequence is the same as the reference sequence or has a specified percentage of nucleotides or amino acid residues that are the same at the corresponding locations within the reference sequence when the two sequences are optimally aligned. For instance, an amino acid sequence that is “substantially identical” to a reference sequence has at least about 60% identity, preferably 65%, 70%, 75%, 80%, 85%, 90%, 91%, 92%, 93%, 94%, 95%, 96%, 97%, 98%, 99%, or higher percentage identity (up to 100%) to the reference sequence when compared and aligned for maximum correspondence over the full length of the reference sequence as measured using a BLAST or BLAST 2.0 sequence comparison algorithms with default parameters, or by manual alignment and visual inspection (see, e.g., NCBI web site).

### BRIEF DESCRIPTION OF THE DRAWINGS

FIGS. 1A-1E are graphs showing that helper T cell epitope immunodominance is controlled by structure in the outer domain of HIV gp120. FIG. 1A: Proliferative responses of three CBA mice. FIG. 1B: Frequency of response by peptide in ten mice of each group. FIG. 1C: Frequency of response by residue in Balb/c and CBA mice (combined), a group of seven HIV-infected individuals, and in the T-helper map from the HIV Immunology Database. FIG. 1D: Ribbon diagram of HIV gp120. FIG. 1E: Frequency of response in mice aligned with the profile of flexibility from the crystal structure (line graph). Numbers above the profile indicate promiscuously immunodominant peptides.

FIGS. 2A-2D are graphs showing the properties of gp120 disulfide variants. FIG. 2A: Binding of various monoclonal antibodies. FIG. 2B: CD spectra. FIG. 2C: CD4 binding. FIG. 2D: Limited proteolysis with cathepsin S and trypsin. Indicated proteins were treated with cathepsin S for 30 mm at room temperature or with trypsin for the indicated times (mm) on ice and analyzed by non-reducing SDS PAGE followed by staining with Coomassie blue. Filled arrows indicate major fragments. Deletion of the disulfide flanking V3 (gp120dss296) increased the sensitivity to cathepsin S with retention of wild-type fragmentation, whereas deletion of a disulfide flanking V4 (gp120dss378 or gp120dss385) caused changes in the fragmentation pattern. Trypsin is expected to produce the 14 kDa fragment (due to cleavage after residue 432) only when the 378-445 disulfide is reduced or deleted.

FIGS. 3A-3E are graphs showing T and B cell responses and correlations in groups of Balb/c mice immunized with wild-type gp120 or one of the disulfide variants. FIG. 3A: CD4+ T cell epitope frequency analyzed by splenocyte proliferation. FIG. 3B: Antibody titers for binding to wild-type gp120. FIG. 3C: Correlation coefficients for peptide-specific T cell responses (5.1.) with serum antibody binding (A⁴⁵⁰) to gp120 or ENVodc (for each peptide, left and right bars, respectively). Horizontal lines indicate the approximate level where r becomes significant (r=0.6, p=0.05). FIG. 3D: Significant (p<0.05) positive and negative correlations (filled and open boxes, respectively) of peptide-specific T cell responses with serum reactivity with peptide. FIG. 3E: Inhibition of CD4 binding to gp120 by antisera.

FIGS. 4A-4B are graphs showing the local changes in the shape of the CD4+ T cell epitope profiles between gp120dss378 (FIG. 4A) and gp120dss385 (FIG. 4B). The influence of structure on epitope dominance is expected to occur in the stably-folded outer domain (boxed). Brackets indicate the positions of deleted disulfide bonds.

FIGS. 5A-5B are charts showing ELISA with envelope proteins expressed in mammalian tissue culture (FIG. 5A; Cross-reactivity: Viral strain) or with gp120 expressed in insect cells (FIG. 5B; Cross-reactivity: Disulfide variant).

FIGS. 6A-6D are graphs showing the frequency of reactivity to linear antibody epitopes in mouse antisera following immunization with wild-type gp120 (FIG. 6A), or the disulfide variants gp120dss296 (FIG. 6B), gp120dss378 (FIG. 6C), and gp120dss385 (FIG. 6D).

FIG. 7 is a ribbon diagram of T4Hsp10. Protease K cleavage sites are indicated in the mobile loop. The immunodominant epitope (peptide 4, filled in black) lies on the N-terminal flank of the mobile loop.

FIGS. 8A-8D are graphs showing the immunogenic sequences in T4Hsp10 (FIG. 8A) and deletion variants (FIG. 8B-8D). The open bars indicate data from a replicate experiment with T4Hsp10. Several newly immunogenic sequences were observed in the mobile-loop deletion variants. The immunodominant sequence corresponding to peptide 4 remained strongly immunogenic in T4Hsp10dLIG (FIG. 8B) but became much less immunogenic in T4Hsp10d8C (FIG. 8D).

FIG. 9 is a graph showing the cross-reactivity of sera from two mice of each group measured by blocking with the various antigens prior to ELISA with the immunogen (Dai et al., J. Biol. Chem. 277:161 (2002)).

FIG. 10 is a diagram of the human immunodeficiency virus type 1 (HIV-1) envelope protein. HIV-1 envelope has two major domains, gp120 and gp41. Disulfide bonds that from between cysteine resides cause the formation of variable (V) and constant (C) loop regions in gp120. The local of the cysteine residues illustrated here correspond to the HIV_{LAI} isolate; other HIV isolates will have different primary envelope nucleotide and amino acid sequences yet will maintain similar advanced amino acid structures (e.g., secondary, tertiary, and quaternary structures).

### DETAILED DESCRIPTION OF THE INVENTION

This invention features a method of increasing the immunogenicity of an antigen by removing or disrupting one or more disulfide bonds from a folded peptide, polypeptide, or glycoprotein (e.g., HIV-1 envelope gp120). For example, the removal of one or more disulfide bonds from the HIV gp120 glycoprotein altered the T cell and neutralizing antibody responses to the glycoprotein when administered to a mammal. The neutralizing antibody titer was increased when test animals were vaccinated with a gp120 in which at least one disulfide bond was removed or disrupted when compared to corresponding titers recorded in animals that were immunized with unaltered, native structure gp120 molecules. In one embodiment of the invention, the removal of one or more disulfide bonds from an antigen increases its ability to elicit neutralizing antibodies to a pathogenic antigen in a mammal. The production of broad and potent neutralizing antibodies is particularly relevant to the design and production of vaccines to protect against or treat pathogens (e.g., bacteria, fungi, viruses, and parasites).

The invention also features vaccines, peptides, polypeptides, and glycoproteins in which one or more disulfide bonds have been removed or disrupted, for the treatment of or protection of a pathogenic infection. Furthermore, the invention provides expression vectors encoding the sequence of vaccines for the in vitro manufacture of, and in vivo and ex vivo administration of the vaccine agents of the invention. In one embodiment of the invention, the vaccines, expression vectors, and their methods of use are useful for the prevention or treatment of HIV infection in a human.

### Improving the Immunogenicity of an Antigen

The present invention features a method of improving the immunogencity of an antigen (e.g., a pathogenic antigen, such as one derived from a bacterium, fungus, virus, or parasite). Many antigens, including peptide, polypeptide, and glycoprotein antigens have a complex, three-dimensional structure (i.e., tertiary structures) in their natural, native state. Disulfide bridges between thiol groups of cysteine residues in these peptides, polypeptides, and glycoproteins facilitate and support these tertiary structures by forming intrachain bonds that bring the respective cysteine residues into close proximity.

The disulfide bonds of a pathogenic antigen can be removed or disrupted by removing one or more cysteine residues from the polypeptide chain of the antigen. Accordingly, the invention features methods of altering an antigen by deleting or substituting cysteine residues in pathogenic antigens. The deletion or substitution of one or more cysteine residues results in an alteration of the tertiary structure of the pathogenic antigen that improves the immunogenicity of the antigen when administered to a subject (e.g., a human). A disulfide bond can be removed by replacing one or more cysteine residues in the pathogenic antigen with, e.g., alanine residues to remove one or more intrachain disulfide bonds. Any other amino acid substitution for one or more cysteine residues that does not otherwise alter the function of the pathogenic antigen (e.g., receptor binding function) can be made to remove one or more disulfide bonds. Alternatively, a pathogenic antigen can be exposed to a chemical agent (e.g., a reducing agent) capable of removing or disrupting one or more disulfide bonds.

In one embodiment of the invention, cysteine residues that form a variable (V) loop region of the human immunodeficiency virus (HIV) gp120 glycoprotein are removed or disrupted to alter the secondary, tertiary, or quaternary amino acid structures of this molecule. For example, the third (V3) and fourth (V4) variable loops of gp120 are formed by disulfide bonds between cysteine residues. As shown in FIG. 10 for the HIV_{LAI} isolate, disulfide bonds bridging cysteine residues located at positions 301 and 306, and between 390 and 423, form the V3 and V4 loops of gp120, respectively. As the envelope glycoprotein of HIV is prone to mutagenesis during viral replication, the location of cysteine residues that form these structural regions differs between viral species and isolates. Persons having skill in the art can use resources such as the HIV Sequence Database (http://www.hiv.lan1.gov/content/sequence/HIV/mainpage.html) to identify, in a known or newly-identified HIV isolates, relevant cysteine residues that can be removed or disrupted according to the methods of the invention. A skilled artisan can, for example, compare an experimental sequence, whether novel or previously described, with a common HIV lab strain (e.g., HIV_{LAI}) by performing a sequence alignment. This comparison will allow the skilled artisan to determine which cysteine residues pair by disulfide bonding to form the gp120 variable loop regions.

### Vaccines of the Invention

The invention also features vaccines (e.g., peptides, polypeptides, and glycoproteins) that can be used to prophylactically or therapeutically vaccinate a patient infected with or at risk of becoming infected with a pathogenic organism (e.g., a bacterium, fungus, virus, or parasite). When used in vaccine or immunogenic compositions, the vaccines of the invention can be administered as peptidic immunogens, or as the immunogenic component of a recombinant (e.g., subunit) or whole-organism (e.g., whole-virus) vaccine. The invention further features expression vectors that encode the nucleic acid sequence of one or more vaccines of the invention. Expression vectors of the invention can be used to recombinantly express a vaccine in a cell or organism, or can be directly administered to patient infected with, or at risk of becoming infected with, a pathogen.

A vaccine of the invention is an antigen (e.g., peptide, polypeptide, or glycoprotein) derived from a pathogen (e.g., a bacterium, fungus, virus, or parasite) which has been altered by removing or disrupting one or more disulfide bonds. Disulfide bonds can be removed or disrupted by deleting or substituting cysteine residues in recombinantly expressed antigens, or exposing a naturally isolated antigen to a chemical or biological reducing agent capable of reducing or disrupting one or more disulfide bonds.

The protein structure (e.g., secondary, tertiary, or quarternary structure) of an antigen for use as a vaccine of the invention is altered by the removal or disruption of one or more disulfide bonds can be a whole protein, or a fragment of a protein (e.g., a domain or splice variant). An antigen of the invention can be 5, 10, 15, 20, 25, 30, 40, 50, 100, 200, 300, 400, 500 or more amino acids in length. An antigen of the invention can have 1, 2, 3, 4, 5, 6, 7, 8, 9, 10 or more disulfide bonds removed relative to the wild-type (unaltered) state of the antigen. In one embodiment, a single disulfide bond is removed relative to the unaltered antigen.

Vaccines of the invention (e.g., peptides, polypeptides, and glycoproteins) for use in eliciting immune responses against a pathogen (e.g., a bacterium, fungus, virus, or parasite) can be prepared recombinantly in vitro or in vivo using any of the expression vectors described herein with an appropriate cell line (e.g., a bacterial, fungal, or animal (e.g., mammalian) cell line). When produced recombinantly, one or more cysteine residues responsible for the formation of one or more disulfide bonds can be deleted or substituted with different amino acid residues (e.g., alanine residues). Alternatively, one or more disulfide bonds in a vaccine, produced recombinantly or naturally isolated, can be disrupted by exposing the vaccine to a disulfide bond reducing agent. Disulfide bond reducing agents include chemical agents, such as dithiothreitol or glutathione, and biological agents, such as protein disulfide isomerase (PDI) and thioredoxin. The strength of the reducing agent, as well as the length of the exposure of the vaccine to the reducing conditions can be used by the skilled artisan to modulate the disruption of disulfide bonds by this method.

The vaccines disclosed in this invention can be prepared conventionally by chemical synthesis techniques, such as described by Merrifield, J. Amer. Chem. Soc. 85:2149 (1963) (see also, e.g., Stemmer et al., 164 Gene 49 (1995)). For example, the vaccines can be readily prepared using solid phase peptide synthesis (SPPS). Automated solid phase synthesis can be performed using any one of a number of well-known, commercially available automated synthesizers, such as the Applied Biosystems ABI 433A peptide synthesizer. Vaccines produced synthetically can, as with recombinant production, be produced with a reduced number of cysteine residues (e.g., deletion or substitution) or one or more disulfide bonds can be removed by exposure to a chemical or biological reducing agent.

Vaccines of the invention (e.g., peptides, polypeptides, or glycoproteins) can also be isolated from a natural source (e.g., from a pathogen, such as a bacterium, fungus, virus, or a parasite). Exposure of a naturally produced pathogenic antigen to a reducing agent (e.g., dithiothreitol or glutathione) can be used to disrupt one or more disulfide bonds to allow use as a vaccine of the invention.

### Expression Vectors of the Invention

The invention also features expression vectors having nucleotide sequences (e.g., DNA or RNA) that encode one or more vaccines of the invention. The expression vector can be a carrier (e.g., a liposome), a plasmid, a cosmid, a yeast artificial chromosome or a virus that includes a nucleotide sequence encoding the vaccine of the invention. The expression vector may include nucleic acid sequences from several sources.

Expression vectors encoding a vaccine of the invention can be constructed using any recombinant molecular biology technique known in the art. The expression vector, upon transfection or transduction of a target cell, can be extrachromosomal or it can be integrated into the host cell chromosome. The nucleic acid component of an expression vector can be in single or multiple copy number per target cell, and can be linear, circular, or concatamerized.

Expression vectors of the invention can also include internal ribosome entry site (IRES) sequences to allow for the expression of multiple peptide or polypeptide chains from a single nucleic acid transcript. For example, an expression vector of the invention can encode a vaccine of the invention as well as another polypeptide (e.g., a detectable label, such as green fluorescent protein (GFP)).

Expression vectors of the invention further include gene expression elements that facilitate the expression of a vaccine of the invention. Gene expression elements useful for the expression of an expression vector encoding a vaccine of the invention include, but are not limited to (a) regulatory sequences, such as viral transcription promoters and their enhancer elements, such as the SV40 early promoter, Rous sarcoma virus LTR, and Moloney murine leukemia virus LTR; (b) splice regions and polyadenylation sites such as those derived from the SV40 late region; and (c) polyadenylation sites such as in SV40. Also included are plasmid origins of replication, antibiotic resistance or selection genes, multiple cloning sites (e.g., restriction enzyme cleavage loci), and other viral gene sequences (e.g., sequences encoding viral structural, functional, or regulatory elements, such as the HIV long terminal repeat (LTR)).

### Ex Vivo Transfection and Transduction

The invention also features methods for the ex vivo transfection and transduction of cells (e.g., blood cells, such as lymphocytes) derived from a donor subject (e.g., a human). Cells from a donor subject can be transfected or transduced ex vivo with an expression vector encoding the nucleotide sequence of a vaccine of the invention to allow for the temporal or permanent expression of the vaccine in the treated subject. Upon administering these modified cells back to the donor subject, the vaccine of the invention will be expressed and can elicit protective or therapeutic immune responses (e.g., neutralizing antibody responses) directed against the immunogen.

Several types of expression vector can be employed to deliver a nucleotide sequence encoding a vaccine of the invention to a cell (e.g., a blood cell, such as a lymphocyte). Expression vectors of the invention include viruses, naked DNA, oligonucleotides, cationic lipids (e.g., liposomes), cationic polymers (e.g., polysomes), virosomes, and dendrimers. The present invention provides for the ex vivo transfection or transduction of cells (e.g., blood cells) followed by administration of these cells back into the donor subject to allow for the expression of a vaccine of the invention that has immunogenic properties. Cells that can be isolated and transfected or transduced ex vivo according to the methods of invention include, but are not limited to, blood cells, skin cells, fibroblasts, endothelial cells, skeletal muscle cells, hepatocytes, prostate epithelial cells, and vascular endothelial cells. Stem cells are also appropriate cells for expression of a vaccine of the invention. Totipotent, pluripotent, multipotent, or unipotent stem cells, including bone marrow progenitor cells and hematopoietic stem cells (HSC), can be isolated, transfected or transduced with an expression vector encoding a vaccine of the invention, and administered to a subject according to the methods of the invention.

The method of transfection or transduction used to express a vaccine of the invention has a strong influence on the strength and longevity of protein expression in the transfected or transduced cell, and subsequently, in the subject receiving the cell. The present invention provides vectors that are temporal (e.g., adenoviral vectors) or long-lived (e.g., retroviral vectors) in nature. Regulatory sequences (e.g., promoters and enhancers) are known in the art that can be used to regulate protein expression. The type of cell being transfected or transduced also has a strong bearing on the strength and longevity of protein expression. For example, cell types with high rates of turnover can be expected to have shorter periods of protein expression.

### Viral Vectors

Viruses encoding the nucleotide sequence of a vaccine of the invention can be used as an expression vector of the invention. The nucleotide sequence of the agent is inserted recombinantly into that of a natural or modified (e.g., attenuated) viral genome suitable for the transduction of a subject (e.g., in vivo administration) or cells isolated from a subject (e.g., for ex vivo transduction followed by administration of the cells back to the subject). Additional modifications can be made to the virus to improve infectivity or tropism (e.g., pseudotyping), reduce or eliminate replicative competency, or reduce immunogencity of the viral components (e.g., all components not related to the immunogenic vaccine agent). The vaccine of the invention can be expressed by the transduced cell and secreted into the extracellular space or remain with the expressing cell (e.g., as an intracellular molecule or displayed on the cell surface). Chimeric or pseudotyped viral vectors can also be used to transduce a cell to allow for expression of a vaccine of the invention. Exemplary viruses are described below.

### Adenoviruses

Recombinant adenoviruses offer several significant advantages for use as expression vectors for the expression of a vaccine of the invention. The viruses can be prepared to high titer, can infect non-replicating cells, and can confer high-efficiency transduction of target cells ex vivo following contact with a target cell population. Furthermore, adenoviruses do not integrate their DNA into the host genome. Thus, their use as expression vectors has a reduced risk of inducing spontaneous proliferative disorders. In animal models, adenoviral expression vectors have generally been found to mediate high-level expression for approximately one week. The duration of transgene expression (expression of a nucleic acid encoding a vaccine of the invention) can be prolonged by using cell or tissue-specific promoters. Other improvements in the molecular engineering of the adenoviral expression vector itself have produced more sustained transgene expression and less inflammation. This is seen with so-called “second generation” vectors harboring specific mutations in additional early adenoviral genes and “gutless” expression vectors in which virtually all the viral genes are deleted utilizing a Cre-Lox strategy (Engelhardt et al., Proc. Natl. Acad. Sci. USA 91:6196 (1994) and Kochanek et al., Proc. Natl. Acad. Sci. USA 93:5731 (1996), each herein incorporated by reference).

### Adeno-Associated Viruses (AAV)

Adeno-associated viruses (rAAV), derived from non-pathogenic parvoviruses, can also be used to express a vaccine of the invention as these vectors evoke almost no anti-vector cellular immune response, and produce transgene expression lasting months in most experimental systems.

### Retroviruses

Retroviruses are useful for the expression of a vaccine of the invention in a cell (e.g., a blood cell, such as a lymphocyte). Unlike adenoviruses, the retroviral genome is based in RNA. When a retrovirus infects a cell, it will introduce its RNA together with several enzymes into the cell. The viral RNA molecules from the retrovirus will produce a double-stranded DNA copy, called a provirus, through a process called reverse transcription. Following transport into the cell nucleus, the proviral DNA is integrated in a host cell chromosome, permanently altering the genome of the transduced cell and any progeny cells that may derive from this cell. The ability to permanently introduce a gene into a cell or organism is the defining characteristic of retroviruses used for gene therapy. Retroviruses include lentiviruses, a family of viruses including human immunodeficiency virus (HIV) that includes several accessory proteins to facilitate viral infection and proviral integration. Current, “third-generation” lentiviral vectors feature total replication incompetence, broad tropism, and increased gene transfer capacity for mammalian cells (see, e.g., Mangeat and and Trono, Human Gene Therapy 16(8):913 (2005) and Wiznerowicz and Trono, Trends Biotechnol. 23(1):42 (2005), each herein incorporated by reference).

### Other Viral Vectors

Besides adenoviral and retroviral vectors, other viral vectors and techniques are known in the art that can be used to express a vaccine of the invention in a cell (e.g., a blood cell, such as a lymphocyte). Theses viruses include Poxviruses (e.g., vaccinia virus (see, e.g., U.S. Pat. Nos. 4,603,112 and 5,762,938, each herein incorporated by reference)), Herpesviruses, Togaviruses (e.g., Venezuelan Equine Encephalitis virus (see, e.g., U.S. Pat. No. 5,643,576, herein incorporated by reference)), Picornaviruses (e.g., poliovirus (see, e.g., U.S. Pat. No. 5,639,649, herein incorporated by reference)), Baculoviruses, and others described by Wattanapitayakul and Bauer (Biomed. Pharmacother. 54:487 (2000), herein incorporated by reference.

### Other Expression Vectors: Naked DNA and Oligonucleotides

Naked DNA or oligonucleotides encoding a vaccine of the invention can also be used to express a vaccine of the invention in a cell (e.g., a blood cell, such as a lymphocyte). See, e.g., Cohen, Science 259:1691-1692 (1993); Fynan et al., Proc. Natl. Acad. Sci. USA, 90:11478 (1993); and Wolff et al., BioTechniques 11:474485 (1991), each herein incorporated by reference. This is the simplest method of non-viral transfection. Efficient methods for delivery of naked DNA exist such as electroporation and the use of a “gene gun,” which shoots DNA-coated gold particles into a cell using high pressure gas and carrier particles (e.g., gold).

### Lipoplexes and Polyplexes

To improve the delivery of an expression vector encoding a vaccine of the invention into a cell, lipoplexes (e.g., liposomes) and polyplexes can be used to protect the expression vector DNA from undesirable degradation during the transfection process. Plasmid DNA can be covered with lipids in an organized structure like a micelle or a liposome. When the organized structure is complexed with DNA it is called a lipoplex. There are three types of lipids, anionic (negatively-charged), neutral, or cationic (positively-charged). Lipoplexes that utilize cationic lipids have proven utility for gene transfer. Cationic lipids, due to their positive charge, naturally complex with the negatively-charged DNA. Also as a result of their charge they interact with the cell membrane, endocytosis of the lipoplex occurs, and the DNA is released into the cytoplasm. The cationic lipids also protect against degradation of the DNA by the cell.

Complexes of polymers with DNA are called polyplexes. Most polyplexes consist of cationic polymers and their production is regulated by ionic interactions. One large difference between the methods of action of polyplexes and lipoplexes is that polyplexes cannot release their DNA load into the cytoplasm, so to this end, co-transfection with endosome-lytic agents (to lyse the endosome that is made during endocytosis) such as inactivated adenovirus must occur. However, this is not always the case; polymers such as polyethylenimine have their own method of endosome disruption as does chitosan and trimethylchitosan.

Exemplary cationic lipids and polymers that can be used in combination with an expression vector of the invention to form lipoplexes, or polyplexes include, but are not limited to, polyethylenimine, lipofectin, lipofectamine, polylysine, chitosan, trimethylchitosan, and alginate.

### Hybrid Methods

Several hybrid methods of gene transfer combine two or more techniques. Virosomes, for example, combine lipoplexes (e.g., liposomes) with an inactivated virus. This approach has been shown to result in more efficient gene transfer in respiratory epithelial cells than either viral or liposomal methods alone. Other methods involve mixing other viral vectors with cationic lipids or hybridising viruses. Each of these methods can be used to facilitate transfer of an expression vector encoding a vaccine of the invention into a cell (e.g., a blood cell, such as a lymphocyte).

### Dendrimers

Dendrimers may be also be used to transfer an expression vector encoding a vaccine of the invention into a cell (e.g., a blood cell, such as a lymphocyte). A dendrimer is a highly branched macromolecule with a spherical shape. The surface of the particle may be functionalized in many ways, and many of the properties of the resulting construct are determined by its surface. In particular it is possible to construct a cationic dendrimer (i.e. one with a positive surface charge). When in the presence of genetic material such as DNA or RNA, charge complimentarity leads to a temporary association of the nucleic acid with the cationic dendrimer. On reaching its destination the dendrimer-nucleic acid complex is then taken into the cell via endocytosis.

### In Vivo Administration

The invention also features in vivo methods for immunizing a subject (e.g., a human) with a vaccine of the invention. In one embodiment, one or more vaccine s (e.g., a peptide, polypeptide, or glycoprotein) of the invention can be directly administered to a subject to elicit a protective or therapeutic immune response (e.g., neutralizing antibodies) against a pathogen (e.g., a bacterium, fungus, virus, or parasite). Alternatively, an expression vector encoding a vaccine of the invention, as described above, can be directly administered to a subject to prevent or treat a pathogenic infection. An expression vector (e.g., a viral vector) that efficiently transfects or transduces one or more cells in vivo can elicit a broad, durable, and potent immune response in the treated subject. Upon transfer of the nucleic acid component of the expression vector into a host cell (e.g., a blood cell, such as a lymphocyte), the host cell produces and displays or secretes the vaccine of the invention, which then serves to activate components of the immune system such as antigen-presenting cells (APCs), T cells, and B cells, resulting in the establishment of immunity.

### Pharmaceutical Compositions

The invention features vaccines of the invention (e.g., peptides, polypeptides, and glycoproteins) and expression vectors encoding the vaccines of the invention in combination with one or more pharmaceutically acceptable excipients, diluents, buffers, or other acceptable carriers. The formulation of a vaccine of the invention will employ an effective amount of the peptide, polypeptide, or glycoprotein immunogen. That is, there will be included an amount of antigen which will cause the subject (e.g., a human) to produce a specific and sufficient immunological response so as to impart protection to the subject from subsequent exposure to a pathogen (e.g., a bacterium, fungus, virus, or parasite) or to treat an existing pathogenic infection. For example, a formulation of a vaccine of the invention can contain an amount of antigen which will cause the subject to produce specific antibodies (e.g., neutralizing antibodies) which can confer a protective or therapeutic benefit to the subject. A vaccine or expression vector of the invention can be directly administered to a subject, either alone or in combination with any pharmaceutically acceptable carrier, salt or adjuvant known in the art.

Pharmaceutically acceptable salts may include non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid phosphoric acid, or the like. Metal complexes include zinc, iron, and the like. One exemplary pharmaceutically acceptable carrier is physiological saline. Other physiologically acceptable carriers and their formulations are known to one skilled in the art and described, for example, in Remington's Pharmaceutical Sciences, (18^{th} edition), ed. A. Gennaro, 1990, Mack Publishing Company, Easton, Pa.

Pharmaceutical formulations of a prophylactically or therapeutically effective amount of a vaccine or expression vector of the invention can be administered orally, parenterally (e.g., intramuscular, intraperitoneal, intravenous, or subcutaneous injection, inhalation, intradermally, optical drops, or implant), nasally, vaginally, rectally, sublingually, or topically, in admixture with a pharmaceutically acceptable carrier adapted for the route of administration. The concentration of a vaccine of the invention in the formulation can vary from about 0.1-100 wt. %.

Formulations for parenteral administration of compositions containing a vaccine or expression vector of the invention include sterile aqueous or non-aqueous solutions, suspensions, or emulsions. Examples of suitable vehicles include propylene glycol, polyethylene glycol, vegetable oils, gelatin, hydrogenated naphalenes, and injectable organic esters, such as ethyl oleate. Such formulations may also contain adjuvants, such as preserving, wetting, emulsifying, and dispersing agents. Biocompatible, biodegradable lactide polymer, lactide/glycolide copolymer, or polyoxyethylene-polyoxypropylene copolymers may be used to control the release of the compounds. Other potentially useful parenteral delivery systems for compositions containing a vaccine of the invention include ethylene-vinyl acetate copolymer particles, osmotic pumps, implantable infusion systems, and liposomes.

Liquid formulations can be sterilized by, for example, filtration through a bacteria-retaining filter, by incorporating sterilizing agents into the compositions, or by irradiating or heating the compositions. Alternatively, they can also be manufactured in the form of sterile, solid compositions, which can be dissolved in sterile water or some other sterile injectable medium immediately before use.

Compositions containing a vaccine or expression vector of the invention for rectal or vaginal administration are preferably suppositories which may contain, in addition to active substances, excipients such as coca butter or a suppository wax. Compositions for nasal or sublingual administration are also prepared with standard excipients known in the art. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops or spray, or as a gel.

The amount of active ingredient in the compositions of the invention can be varied. One skilled in the art will appreciate that the exact individual dosages may be adjusted somewhat depending upon a variety of factors, including the peptide being administered, the time of administration, the route of administration, the nature of the formulation, the rate of excretion, the nature of the subject's conditions, and the age, weight, health, and gender of the patient. In addition, the severity of the condition treated by the vaccine will also have an impact on the dosage level. Generally, dosage levels of between 0.1 μg/kg to 100 mg/kg of body weight are administered daily as a single dose or divided into multiple doses. Preferably, the general dosage range is between 250 μg/kg to 5.0 mg/kg of body weight per day. Wide variations in the needed dosage are to be expected in view of the differing efficiencies of the various routes of administration. For instance, oral administration generally would be expected to require higher dosage levels than administration by intravenous injection. Variations in these dosage levels can be adjusted using standard empirical routines for optimization, which are well known in the art. In general, the precise prophylactically or therapeutically effective dosage can be determined by the attending physician in consideration of the above-identified factors.

The amount of a vaccine or expression vector of the invention present in each vaccine dose given to a patient is selected with regard to consideration of the patient's age, weight, sex, general physical condition and the like. The amount of a vaccine or expression vector required to induce an immune response, preferably a protective response (e.g., a neutralizing antibody response), or produce an exogenous effect in the patient without significant adverse side effects varies depending upon the pharmaceutical composition employed and the optional presence of an adjuvant. Initial doses can be optionally followed by repeated boosts, where desirable. The method can involve chronically administering the vaccine or expression vector of the invention. For therapeutic use or prophylactic use, repeated dosages of the immunizing vaccine or expression vector can be desirable, such as a yearly booster or a booster at other intervals. The dosage administered will, of course, vary depending upon known factors such as the pharmacodynamic characteristics of the particular vaccine, and its mode and route of administration; age, health, and weight of the recipient; nature and extent of symptoms, kind of concurrent treatment, frequency of treatment, and the effect desired. A vaccine or expression vector of the invention can be administered in chronic treatments for subjects at risk of acute infection due to needle sticks or maternal infection. A dosage frequency for such “acute” infections may range from daily dosages to once or twice a week i.v. (intravenous) or i.m. (intramuscular), for a duration of about 6 weeks. The vaccine or expression vector can also be employed in chronic treatments for infected patients, or patients with advanced infection with a pathogen (e.g., a virus, bacteria, fungus, or parasite). In infected patients, the frequency of chronic administration can range from daily dosages to once or twice a week i.v. or i.m., and may depend upon the half-life of immunogen of the vaccine or expression vector of the invention.

### Adjuvants

A vaccine of the invention (e.g., peptide, polypeptide, or glycoprotein) used to vaccinate a patient (e.g., a human) in need thereof against a pathogen can be administered concurrent with or in series with one or more pharmaceutically acceptable adjuvants to increase the immunogenicity of the vaccine. Currently, adjuvants approved for human use in the United States include aluminum salts (alum). These adjuvants have been useful for some vaccines including hepatitis B, diphtheria, polio, rabies, and influenza. Other useful adjuvants include Complete Freund's Adjuvant (CFA), Incomplete Freund's Adjuvant (IFA), muramyl dipeptide (MDP), synthetic analogues of MDP, N-acetylmuramyl-L-alanyl-D-isoglutamyl-L-alanine-2-[1,2-dipalmitoyl-s-gly-cero-3-(hydroxyphosphoryloxy)]ethylamide (MTP-PE) and compositions containing a metabolizable oil and an emulsifying agent, wherein the oil and emulsifying agent are present in the form of an oil-in-water emulsion having oil droplets substantially all of which are less than one micron in diameter.

### Kits

The invention provides kits that include a pharmaceutical composition containing a vaccine of the invention, or an expression vector encoding a vaccine of the invention, and a pharmaceutically-acceptable carrier, in a therapeutically effective amount for preventing or treating a pathogenic infection. The kits include instructions to allow a practitioner (e.g., a physician, nurse, or patient) to administer the composition contained therein.

Preferably, the kits include multiple packages of the single-dose pharmaceutical composition(s) containing an effective amount of a vaccine of the invention, or an expression vector encoding a vaccine of the invention. Optionally, instruments or devices necessary for administering the pharmaceutical composition(s) may be included in the kits. For instance, a kit of this invention may provide one or more pre-filled syringes containing an effective amount of a vaccine of the invention, or an expression vector encoding a vaccine of the invention. Furthermore, the kits may also include additional components such as instructions or administration schedules for a patient infected with or at risk of being infected with a pathogenic organism (e.g., a bacterium, fungus, virus, or parasite) to use the pharmaceutical composition(s) containing a vaccine of the invention, or an expression vector encoding a vaccine of the invention.

It will be apparent to those skilled in the art that various modifications and variations can be made in the compositions, methods, and kits of the present invention without departing from the spirit or scope of the invention. Thus, it is intended that the present invention cover the modifications and variations of this invention provided they come within the scope of the appended claims and their equivalents.

### EXAMPLES

The present invention is illustrated by the following examples, which are in no way intended to be limiting of the invention.

### Example 1

### Association of CD4+ Epitopes with Adjacent Flexible Segments in HIV gp120

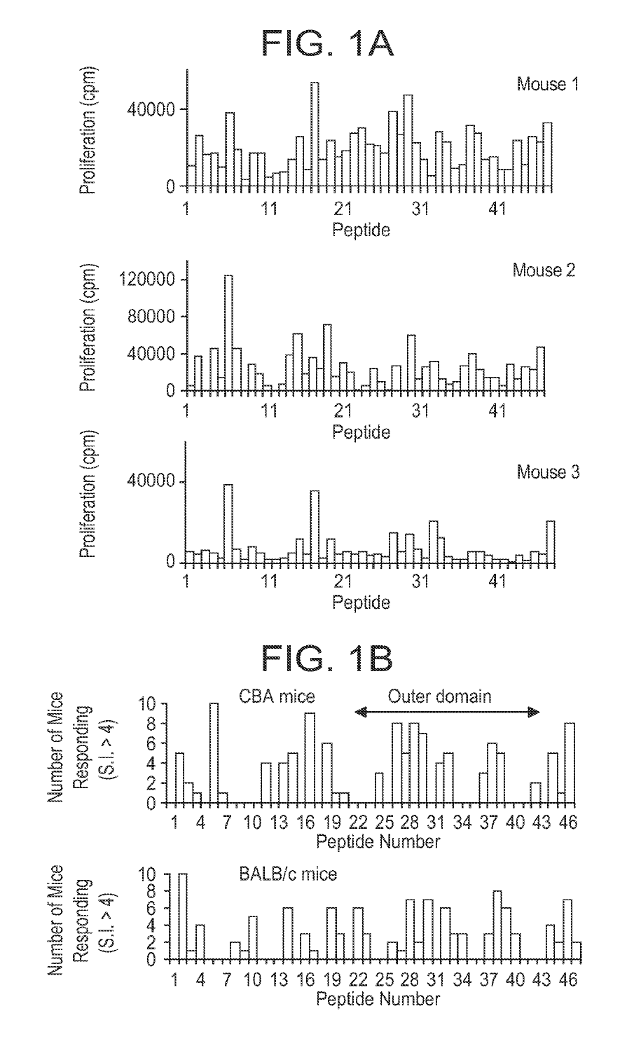

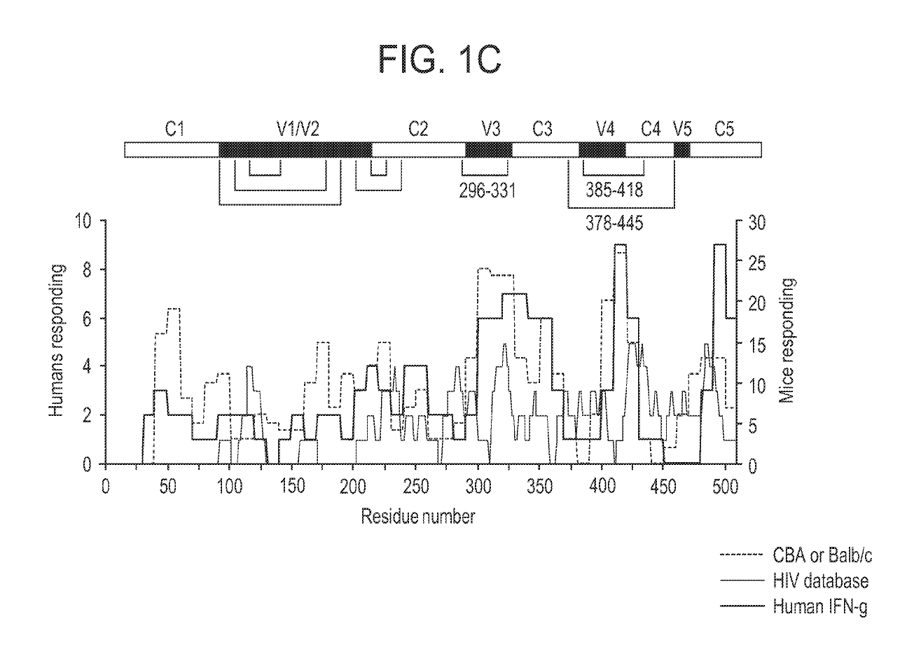

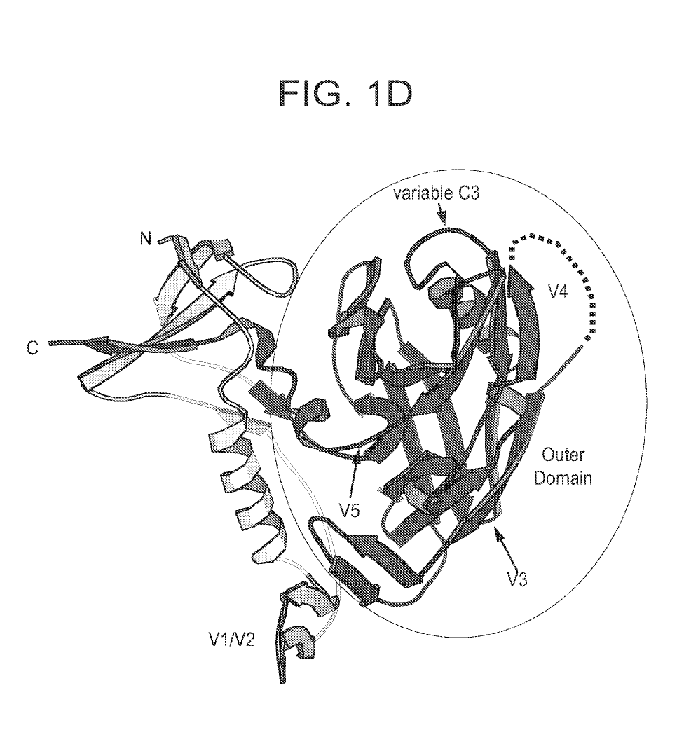

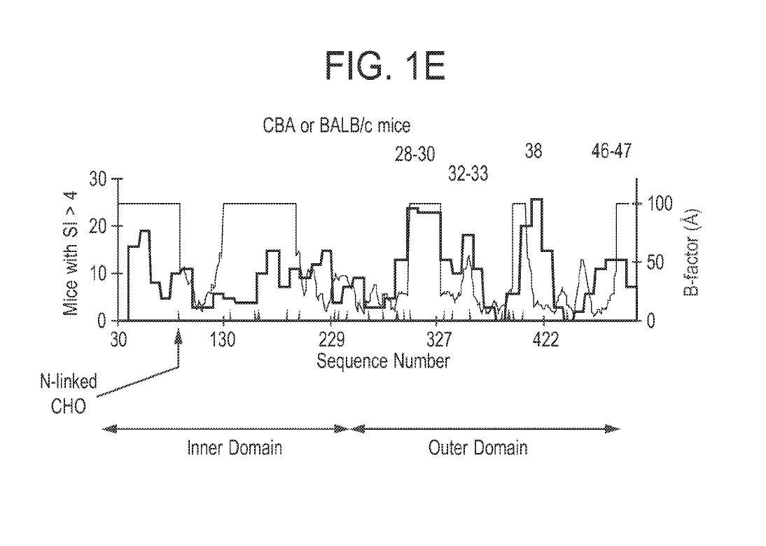

Epitopes were mapped by splenocyte proliferative responses to individual 20-mer peptides that overlapped by 10 residues. Profiles for individual mice differed enormously (FIG. 1A), which creates an opportunity to identify correlations between T cell and antibody responses (see below). In spite of the individual variability, when analyzed for the whole group, certain peptides were most frequently stimulatory and gave the highest levels of proliferation. A majority of mice of both strains responded to promiscuous epitopes clustered in four distinct regions, V3, C3, V4, and C5 despite the presence of different class II MHC alleles in the two strains (FIG. 1B,C). Thus, immunodominance of these epitopes transcends peptide selectivity by the MHC protein.

The association of CD4+ epitopes with flexible segments was strong in the outer domain but weak in the inner domain of gp120 (FIG. 1E). These results can be explained by the relatively unstable three-dimensional structure in the inner domain. In the acidic compartment of the lysosome, the inner domain is likely to lose its three-dimensional structure and therefore expose most of its sequence for binding to MHC proteins. Immunodominant sequences were present in the inner domain, but they were specific to the mouse strain, suggesting that peptide selectivity by the MHC protein was the predominant factor in epitope immunodominance within the inner domain.

CD4+ T cell epitope maps for the outer domain of gp120 are very similar in mucosally immunized mice and HIV-infected humans. The profile of human lymphocytes that secrete interferon gamma (IFNγ) matches the profile of mouse proliferative responses (FIG. 1C). Interestingly, the human IFNγ epitopes tend to be clustered more in the carboxy terminus than do the mouse epitopes, but the strength of the correlation with flexible loops in gp120 is nearly identical. In the mouse profile, epitopes tend to occur eight residues C-terminal from flexible loops characterized by high crystallographic B factor (r²=0.41). In the human profile, epitopes tend to occur sixteen residues C-terminal from flexible loops (r²=0.43).

The profile of T-helper epitopes reported in the HIV Immunology Database (http://www.hivlan1.gov/content/immunology/) corroborates the dominance pattern (FIG. 1C), in that there are similar peaks in the frequency of epitopes associated with flexible loops. However, the Database also indicates that, as a group, humans can prime responses to essentially all outer-domain segments. We hypothesize that the response to some subdominant epitopes may develop too late or with too little intensity to help the development of protective antibodies.

### Example 2

### Poor Immunogenicity of Epitopes Associated with Disulfide Bonds in the Outer Domain

In both gp120-immunized mice and infected humans, sequences on the flanks of the V3 and V4 loops are weakly immunogenic (FIG. 1C). It is possible that the acetamide blocking group on cysteines in some of the synthetic peptides directly interfered with peptide loading or T cell recognition. The naturally processed peptide could have either reduced or oxidized cysteine and therefore the presence of specific T cells could go undetected when stimulating with the acetamide derivative. However, certain weakly immunogenic peptides lack cysteine; and certain other weakly immunogenic peptides have a single cysteine within three residues of the peptide terminus, which is not likely to be at the center of the epitope. Moreover, there was no general defect in loading or recognition of cysteine-containing peptides because 28 of the 47 peptides from gp120 contained cysteine, including many that were strongly immunogenic. Thus, the use of acetamide probably is a good compromise between reduced and oxidized forms. The absence of responses to peptides near the disulfide bonds probably is due to stable structure.

Disulfide-stabilized structure can have a major influence on the dominance pattern of T cell responses. In the endo-lysosome of dendritic cells (DCs), the IFNγ-inducible thiol reductase (GILT) reduces disulfide bonds, but its activity optimum occurs below pH10. The following scenario describes the development of a very narrow T-helper response. A significant fraction of MHC II is loaded with gp120 peptides at an early stage of endo-lysosomal development, wherein the pH is not very low and GILT is less active. Such compartments can be well populated in DCs that are only weakly activated. The gp120 in these compartments would retain disulfide bonds and stable native-like structure. The variable loops would be preferentially cleaved by proteases, but the structure, partially stabilized by disulfide bonds, blocks the exposure of nearby sequences. As a result, a narrow selection of peptides derived from the loops are preferentially loaded into MHC II and presented to T cells.

### Example 3

### Design and Preparation of Disulfide-Bond Variants

Disulfide bonds were individually removed from the outer domain of gp120 (strain 89.6) in order to promote the presentation of nearby epitopes. In each of three variants, a pair of cysteine residues was replaced with a pair of alanine residues (Table I).

TABLE I

| | Disulfide | |
|---|---|---|
| Variant | (HXBc2 numbering) | Location |
| gp120dss296 | 296-331 | Brackets V3 |
| gp120dss378 | 378-445 | Brackets V4, 13 strands |
| | | 20 and 21 |
| gp120dss385 | 385-418 | BracketsV4 |

Disulfide bonds in the outer domain were targeted because stable structure strongly influences helper T cell epitope immunodominance in the outer domain and because poorly immunogenic sequences coincided with disulfide bonds in the outer domain. The outer domain is also where broadly neutralizing antibody epitopes are thought most likely to occur (Wyatt et al., Nature 393:705 (1998)). Only one disulfide bond has been removed in each variant in order to destabilize the three-dimensional structure without destroying it. This could be important for retention of non-neutralizing antibody that reduces gp120 toxicity (Amara et al., J. Virol. 76:6138 (2002)) and for the possible priming of novel neutralizing antibody epitopes. In a number of proteins, removal of a single disulfide bond by alanine substitution was shown to destabilize the protein with retention of native-like structure (Kidera et al., Protein Sci. 3:92 (1994); Ruoppolo et al., Biochem. 39:12033 (2000); and Varallyay et al., Biochem. Biophys. Res. Commun. 230:592 (1997)).

Wild-type gp120 and the three disulfide variants were purified from baculovirus-infected insect cell culture supernatants. In order to increase yield and maintain the native conformation, the proteins were purified using Galanthus nivalis lectin affinity chromatography, followed by nickel-affinity chromatography using the hexahistidine tag incorporated at the C-terminus of the gp120 proteins.

### Example 4

### Recognition of Disulfide Variants by Monoclonal Antibodies and CD4

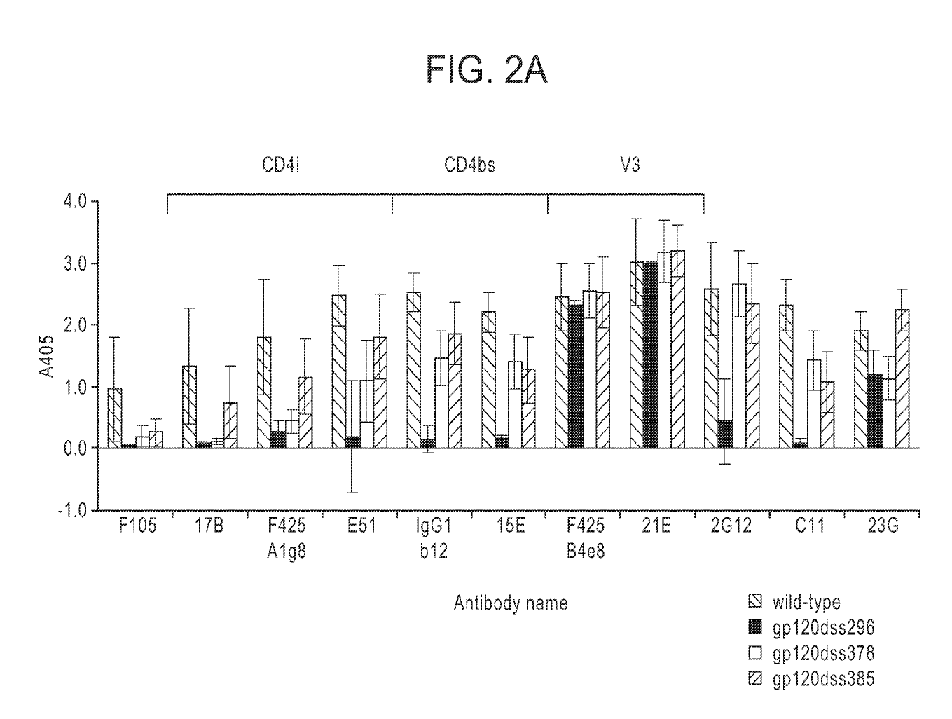

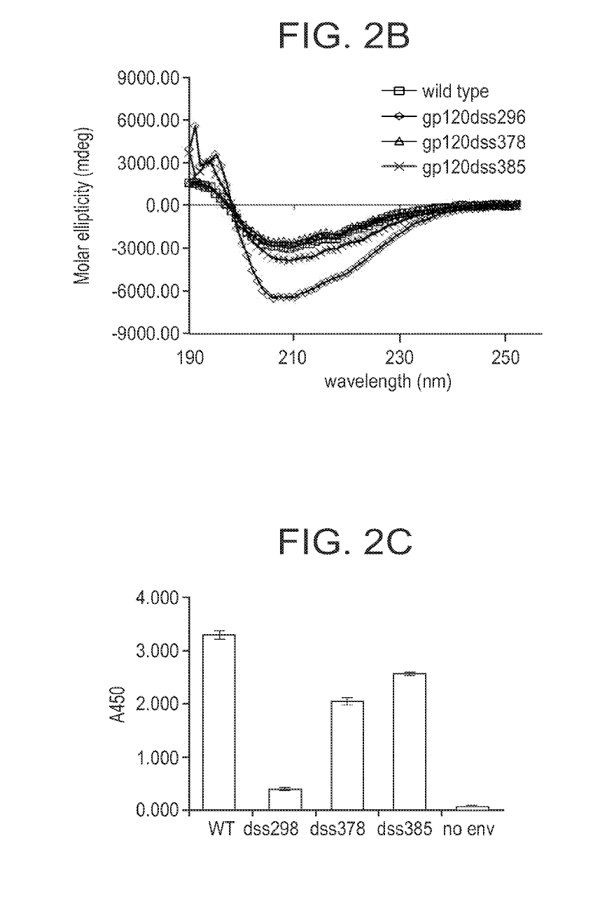

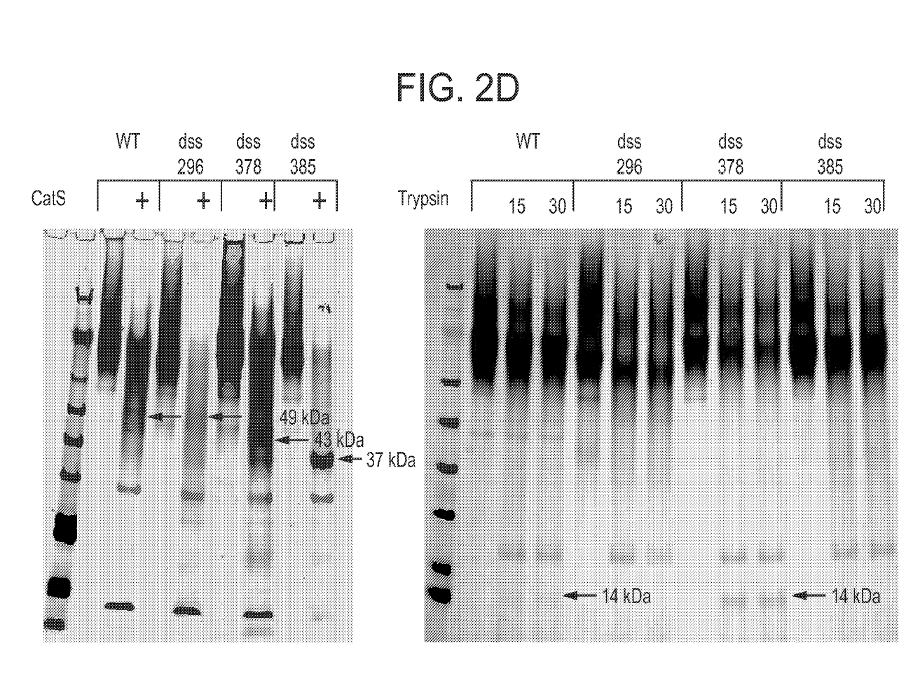

The disulfide deletions altered conformational epitopes and the CD4 binding site in the gp120 variants (FIG. 2A,C). Not surprisingly, all three variants bound V3-specific antibodies (F425-B4e8 and 21E) equally well. gp120dss296 exhibited drastically reduced binding to antibodies specific for conformational epitopes (e.g., b12, 15E, and 2G12), and it also bound CD4 very poorly. In contrast, the other two variants (gp120dss378 and gp120dss385) exhibited substantial reactivity with some, but not all antibodies that recognize three-dimensional epitopes. Both variants bound well to CD4. Of these two variants, gp120dss385 was most similar to wild-type.

### Example 5

### Differences in Circular Dichroism Spectra

Circular dichroism (CD) spectra in the far-ultraviolet region report on the content of regular secondary structure of proteins. As expected for a protein with a high content of beta sheet, the CD spectrum of gp120 has a minimum near 210 nm and the intensity of the CD signal is low (FIG. 2B). The CD spectrum of gp120dss296 is more intense; the minimum has shifted to a shorter wavelength; and a shoulder has appeared at 222 nm. These differences suggest that gp120dss296 contains more alpha helix. The CD spectrum of gp120dss378 is indistinguishable from that of wild-type. The CD spectrum of gp120dss385 indicates a change in secondary structure that is too small to characterize. These results suggest that, with regard to secondary-structure content, gp120dss296 is substantially affected, gp120dss378 is very similar to wild-type, and gp120dss385 is slightly altered.

### Example 6

### Differences in Proteolytic Fragmentation of Disulfide Variants

Changes in gp120 proteolytic fragmentation could have implications for processing of gp120. The effects of deleting disulfide bonds on the structure of gp120 were probed by limited proteolysis and non-reducing SDS-PAGE. Partial digestion by cathepsin S or trypsin indicated that the rank order of resistance to proteolysis was wild-type>gp120dss385>gp120dss378>gp120dss296 (FIG. 2D), which is consistent with the pattern of reactivity with conformational antibodies and CD4 binding. Partial digestion of wild-type gp120 or gp120dss296 with cathepsin S yielded heterogeneous major fragments of approximately 49 kDa, which presumably correspond to a collection of preferred cleavage sites near the middle of the protein. Partial digestion of gp120dss378 or gp120dss385 with cathepsin S yielded more discrete fragments at slightly lower masses (approx. 43 and 37 kDa, respectively), suggesting that preferred cleavage occurs at more C-terminal sites.

Partial digestion with trypsin of wild-type gp120 yielded a small amount of a discrete fragment near 14 kDa, which is consistent with the previously reported cleavage at residue 432, except this fragment should be observed only if the 378-445 disulfide bond is broken. Under the same conditions, digestion of gp120dss296 was more extensive, but the 14-kDa fragment was not evident. Digestion of gp120dss378 was slightly more extensive than wild-type gp120, and the 14-kDa product was much more prominent. This result is consistent with the 14-kDa fragment being released because the 378-445 disulfide bond was deleted. Digestion of gp120dss385 did not yield the 14-kDa fragment, as expected for a protein with an intact 378-445 disulfide bond. Residue 432 is located in the bridging sheet, and trypsin cleavage at this site is prevented by CD4 binding (in the context of gp120 from strain IIIB). Thus, we should be able to determine whether CD4 binding blocks this cleavage in the disulfide variants. A failure to block would suggest a role for the disulfide in stabilizing the bridging sheet.

### Example 7

### Summary of Conformational Analysis

The results from the antibody binding, CD4 binding, CD spectroscopy, proteolysis, and deglycosylation (data not shown) analyses indicate that the removal of disulfide bonds causes distinct changes in structure/flexibility. Removal of the disulfide bond in gp120dss296 globally destabilized and/or altered the conformation of the protein, such that it bound only very weakly to CD4 or antibodies specific for conformational epitopes. Removal of the disulfide bonds in gp120dss378 and gp120dss385 caused more subtle conformational effects. These two variants exhibited reduced binding to CD4 and conformational antibodies, and their proteolytic fragmentation patterns were severe loss of T cell responses

### Example 8

### Broad Reductions in the CD4+ T Cell Responses to Disulfide Variants

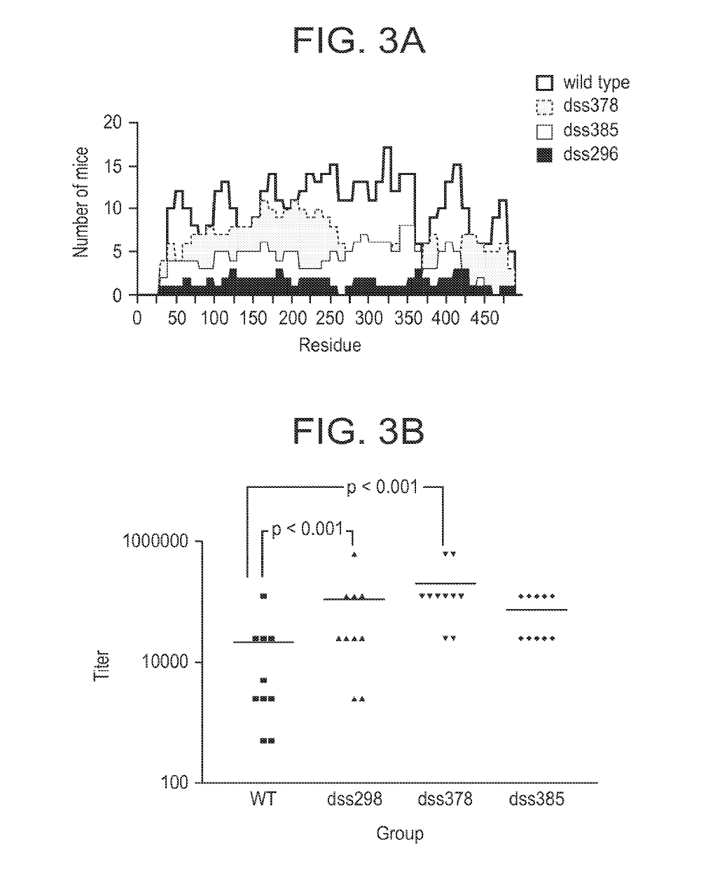

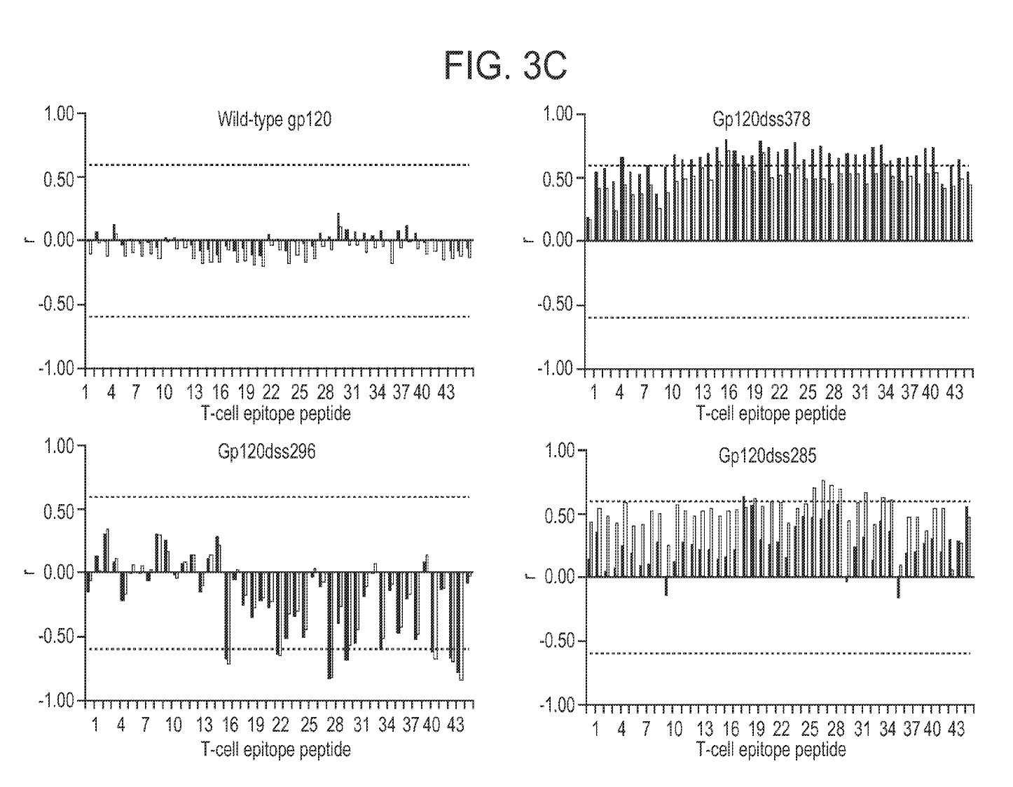

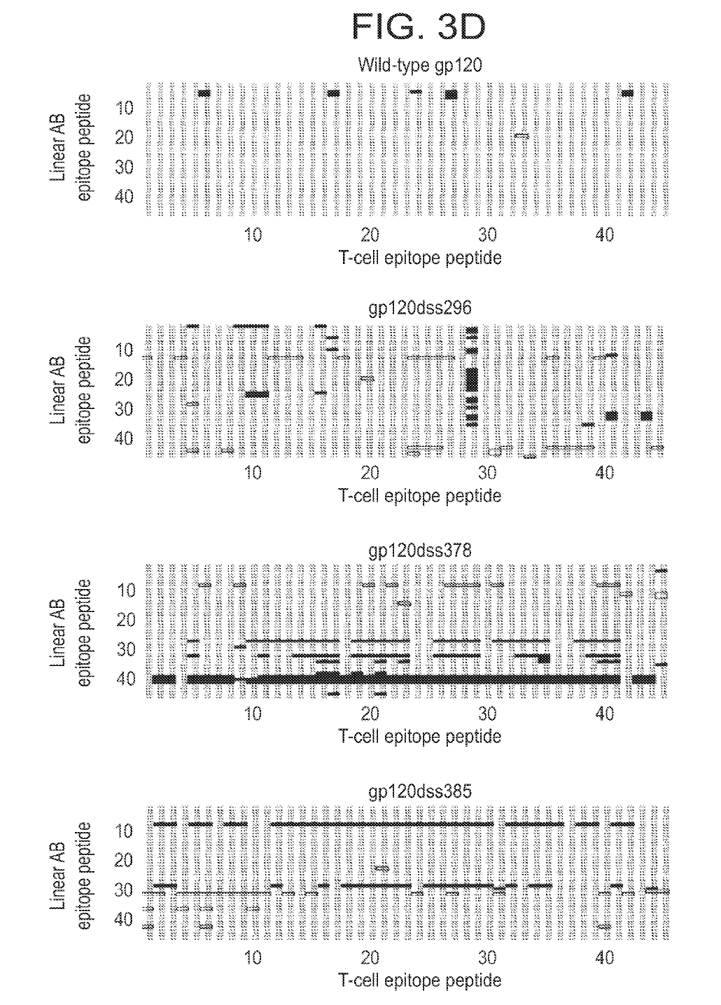

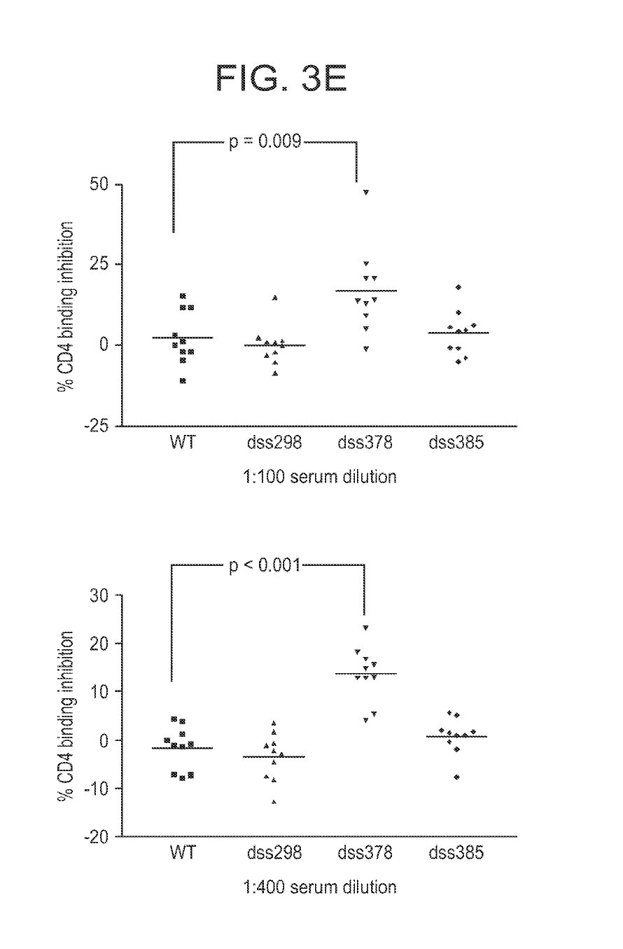

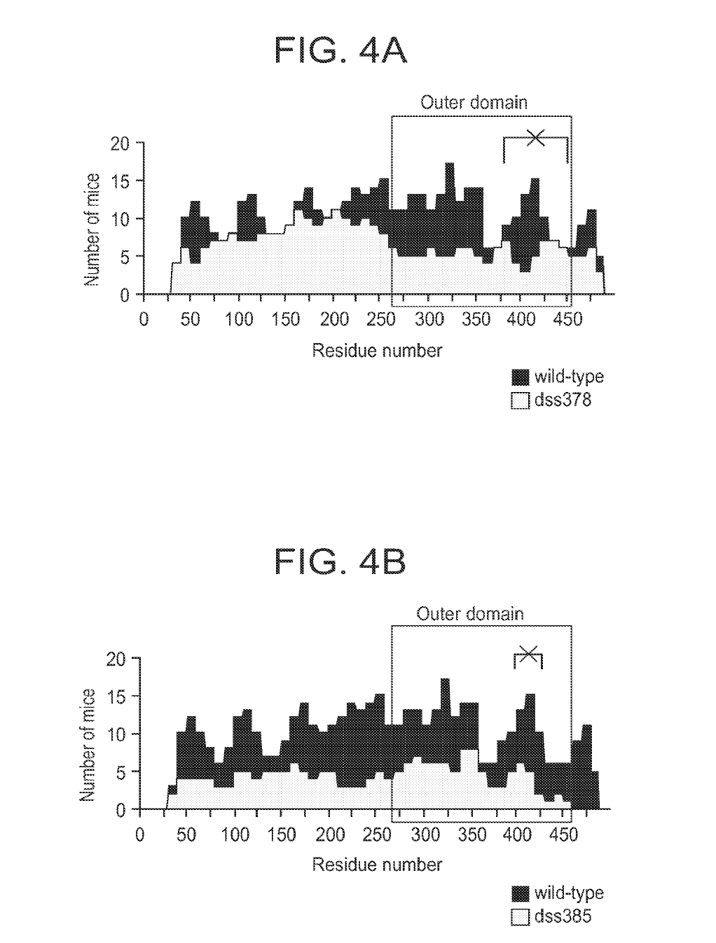

Splenocyte proliferative responses were mapped following immunization of groups of ten Balb/c mice with the gp120 disulfide variants as described above. The deletions in gp120dss296 and gp120dss385 globally reduced the T cell response (FIG. 3A). The deletion in gp120dss378 also reduced the response, but the effect was most pronounced in the outer domain. The broad reductions in the T cell response were not expected. Although the mechanistic basis for the reduction is unclear, our current hypothesis is that it is due to altered pathways of antigen processing. The profile for gp120dss378 exhibited shape differences that was consistent with the expected local increases in flexibility. Deep valleys in the wild-type profile that coincided with the ends of the 378-445 disulfide bond are absent from the profile of the gp120dss378 variant (FIG. 4). In contrast, the profile for gp120dss385 has retained the valleys. The fact that the 385-418 disulfide bond is nested within the loop constrained by the 378-445 disulfide bond may have minimized the local structural impact of its deletion. However, the profile for gp120dss385 indicates a near the C-terminus of gp120, which may be due to tertiary contacts in the three-dimensional structure.

### Example 9

### High Antibody Titers Raised by all Three Disulfide Variants

For the measurement of antibody, antisera were tested for reactivity with wild-type gp120. Two of the variants elicited a significantly higher titer of antibody than wild-type gp120 (FIG. 3B). This finding becomes more significant in light of the fact that the T cell responses were lower against the variants.

### Example 10

### Differences in Cross-Reactivity by Antisera Raised Against Disulfide Variants

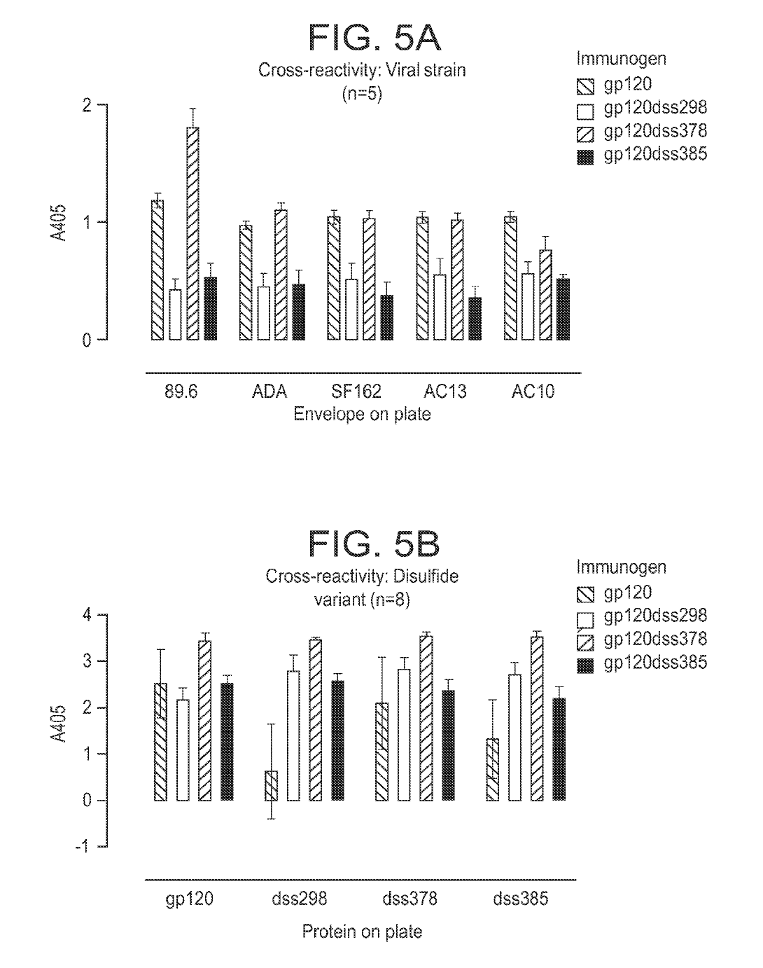

When tested for reactivity with the envelope glycoproteins from other viral strains in a capture assay, the antisera raised against gp120dss378 were comparable to antisera raised against wild-type (FIG. 5). Antisera raised against the other two variants were less cross-reactive. When tested for reactivity with purified wild-type and disulfide variants coated directly onto plates, the antisera raised against gp120dss378 were again the most cross-reactive. These results suggest that antisera raised against gp120dss378 recognize more conformational epitopes than antisera raised against other variants. This conclusion is surprising when considered with results showing that gp120dss385 is more similar to wild-type on the basis of reactivity to monoclonal antibodies and CD4 binding. The especially poor reaction of wild-type antisera with gp120dss296 is consistent with other data suggesting that structure in this variant is substantially altered.

### Example 11

### Differences in Reactivity with Linear Epitopes by Antisera Raised Against Disulfide Variants

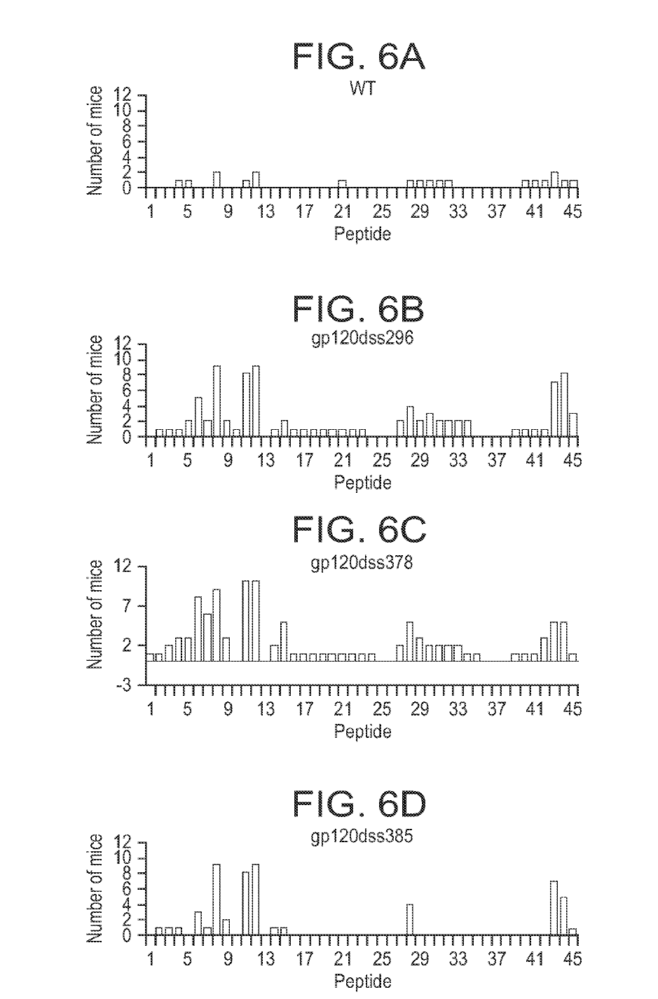

Antisera were tested for reactivity with the panel of 20-mer peptides, which were coated directly onto microtiter wells. A reaction was scored positive if the absorbance was two-times higher than the average background reaction for uncoated wells. The profiles of reactivity for the four different immunogens have virtually the same shape, suggesting that their three-dimensional structures are similar insofar as the display of unstructured segments (FIG. 6). As expected, most reactivity is concentrated in the V1/V2 regions, with smaller clusters of epitopes near V3 and VS. However, additional peptides were reactive at lower frequencies in the profiles for the immunogens that raised higher-titer antibodies, gp120dss296 and gp120dss378.

### Example 12

### Correlation of Serum Reactivity with Peptide-Specific T Cell Responses in the Variants, but not Wild-Type gp120

A major goal of this project involves the identification of T-B collaboration, i.e., correlations of T cell and antibody responses. For the analysis of correlations, the antisera were tested for reaction with wild-type gp120, a recombinant V3, V4-deleted outer domain (ENVodc), and each of the gp120 peptides. ENVodc is similar to the OD1 protein described by Sodroski and coworkers (Yang et al., J. Virol. 78:12975 (2004)), except that ENVodc lacks V3 and V4.

Unexpectedly, there was a near-total lack of correlation between T cell and antibody responses to the wild-type gp120. This is in contrast to the distinct and clustered correlation of responses elicited by immunization with disulfide variants (discussed below). For the immunization with wild-type gp120, antibody reactions with neither gp120 nor ENVodc correlated with any of the T cell responses (FIG. 3C). Moreover, reactions with only a few inner-domain peptides correlated with T cell responses (FIG. 3D).

For gp120dss296, T cell responses to a distinct selection of peptides and—correlated with reactivity to both gp120 and ENVodc. The strongest anti-correlation with reactivity to intact protein involved the T cell response to peptide 29. In terms of antibody reactivity with peptides, there were isolated positive and negative correlations distributed over the molecule and a string of positive correlations with the T cell response to peptide 29 (appearing as a vertical stripe of filled boxes in FIG. 3D). The positive correlations in reactivity to peptides contrast with the negative correlations in reactivity to intact proteins. These results suggest that the antibody specificity may be dominated by linear epitopes and that T cells specific for peptide 29 (corresponding to V3) have an exceptionally strong role in raising antibody against this variant conformational epitope than antisera raised against the other variants.

For gp120dss378, T cell responses broadly correlated with antibody reactivity to gp120 and ENVodc (FIG. 3C). In terms of reactivity to peptides, the pattern of correlations differs from that for gp120dss296 in that correlations appear in horizontal stripes, indicating that the T cell response to many peptides correlates with antibody reactivity to certain peptides (FIG. 3D). Essentially all of the positive correlations involve antibody reactions with peptides in the outer domain (peptides 23-45).

For gp120dss385, T cell responses generally exhibited better correlation with antibody reactivity to ENVodc than with reactivity to full-length gp120, and the best correlations involved T cell responses to peptides from the V3 loop (FIG. 3C). In terms of reactivity to peptides, horizontal stripes of correlation involve antibody reactivity to peptides in V3 (FIG. 3D). Thus, the development of antibody specific for the outer domain in this variant involves T cell and antibody responses to V3.

### Example 13

### Effects of Antisera on CD4 Binding

The antisera were tested for the ability to block CD4 binding to gp120 in order to gauge the potential for the disulfide variants to elicit neutralizing antibodies. Significant inhibition of CD4 binding was observed for antisera raised against only gp120dss378 (FIG. 3E). Inhibition was observed at the lowest dilutions, indicating that the competing antibody is a minor fraction of the total antibody in the antisera. Nevertheless, this level of competition by antisera is comparable to that observed for rabbit antisera raised against gp120 or trimeric envelope glycoproteins (Dey et al., J. Virol. 81:5579 (2007)).

### Example 14

### Viral Neutralization

Using the viral-entry assay with TZM cells (Wei et al., “Nature 422:307 (2003) and Wei et al., Antimicrob. Agents Chemother. 46:1896 (2002), each herein incorporated by reference), two-fold dilutions of antisera from mice immunized with gp120, gp120dss296, gp120dss378, or adjuvant alone were tested for neutralization of pseudovirus that express the 89.6 ENV. The “background” neutralization by sera from mice immunized with adjuvant-only was very high. The average titer for 50% neutralization by wild-type and gp120dss378 antisera was 1:100 (10 antisera each), and it was approximately the same as the adjuvant-only controls (3 antisera). However, the average titer for 50% neutralization by the gp120dss296 antisera was 1:400 (10 antisera), and the level of inhibition was significantly greater than obtained with the other immunogens or controls. This result was surprising in view of the higher antibody titer in gp120dss378 antisera and the appearance of CD4 binding inhibition only in the gp120dss378 antisera. This result suggested that we should not disregard gp120dss296 as a potential vaccine. Although the activity was weaker, neutralization by gp120dss378 antisera also was significantly greater than the controls at several dilutions. It seems likely that specific neutralizing activity is being masked by a non-specific component in the mouse serum.

### Example 15

### Conclusions from Work on HIV gp120

Deletion of conserved disulfides in gp120 caused distinct and dramatic changes in the shape and intensity of the T cell response. Two of the three disulfide deletions (in gp120dss296 and gp120dss378) caused significant increases in antibody titer. The profiles of linear-epitopes induced by all four immunogens were similar in shape, but the high-titer responses were associated with the recruitment of additional low-frequency epitopes. It is possible to identify correlations between specific antibody and specific T cell responses, and the patterns of correlation reflect differences in global conformational stability. We propose that individual correlations (positive or negative) are related to the specificity of the antibodies and alternate pathways of antigen processing and presentation (possibly involving the alternate pathways of disulfide-bond reduction). In the proposed project, similar studies will endeavor to correlate specific T cell responses with protective (CD4bs, viral inhibitory) and non-protective (linear epitope) antibodies and to identify structural modifications that favor development of protective antibodies.

### Example 16

### Parallel Observations with a Model Antigen: Bacteriophage T4 Hsp10

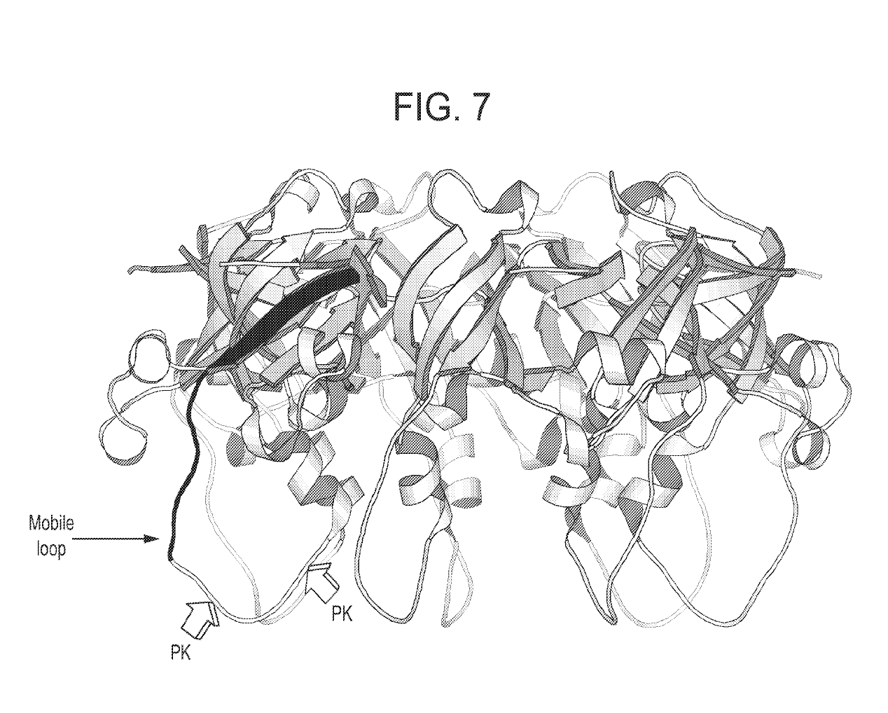

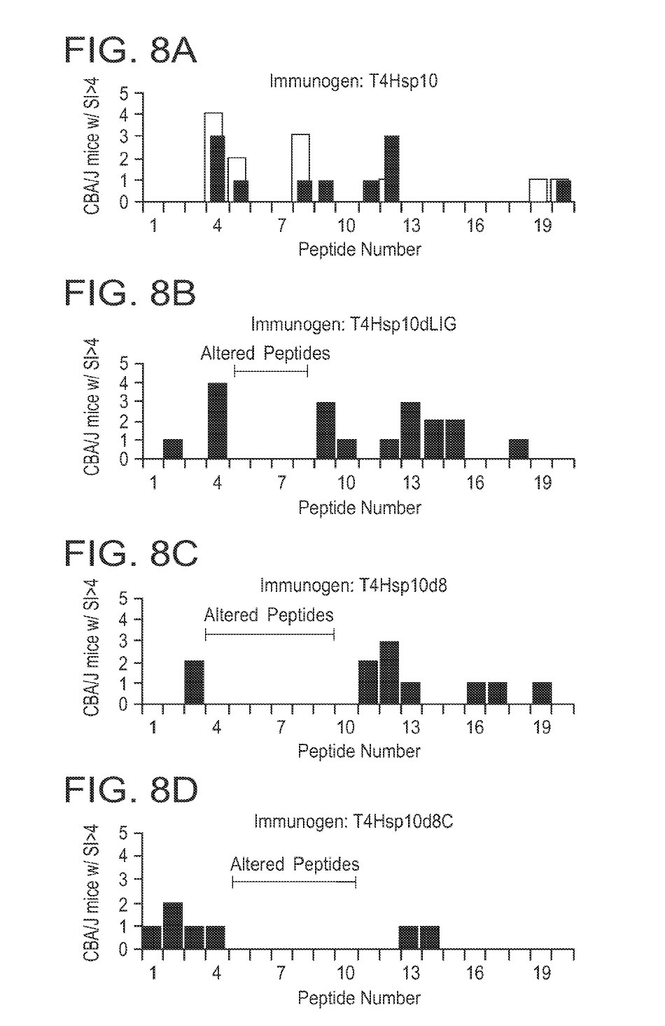

In studies on structural variants of bacteriophage T4Hsp10, we also observed reduced T cell responses, increased antibody titers, and shifts in specificity of both T cell and antibody responses. Helper T cell epitopes in T4Hsp10 were mapped by restimulation of splenocytes from CBA and C57Bl/6 mice immunized in conjunction with mLT. Immunodominant epitopes (peptides 4 and 8) were identified on the N- and C-terminal flanks of a large protease-sensitive mobile loop that is conserved in the structure of all Hsp10s (FIGS. 7 and 8).

Segments of the mobile loop were deleted in order to test the hypothesis that a reduction in its proteolytic sensitivity would shift the epitope immunodominance. We reasoned that if the deletions simultaneously eliminated sensitivity to several proteases, then the loop had lost ability to conform to protease active sites. In the deletion variant T4Hsp10dLIG, three residues were removed from the middle of the loop. In T4Hsp10d8 and T4Hsp10d8C, eleven residues were removed from the middle and C-terminal side of the mobile loop, respectively. The variants all became more resistant to proteolysis and more thermostable. The changes were not due to changes in overall three-dimensional structure, according to analysis of circular dichroism, thermal denaturation, and chemical crosslinking. One of the larger deletions produced the most resistant variant. T4Hsp10d8C became much more resistant to proteolysis, and this may be explained by an increase in secondary structure in the shortened mobile loop. According to circular dichroism spectroscopy, T4Hsp10d8C acquired additional beta-sheet content and exhibited the largest increase in thermostability.

The immunogenicity of the dominant epitope adjacent to the mobile loop (peptide 4) was substantially reduced in T4Hsp10d8C (FIG. 8). In addition, all of the deletion variants induced T cell responses to epitopes that were not observed for the wild-type T4Hsp10.

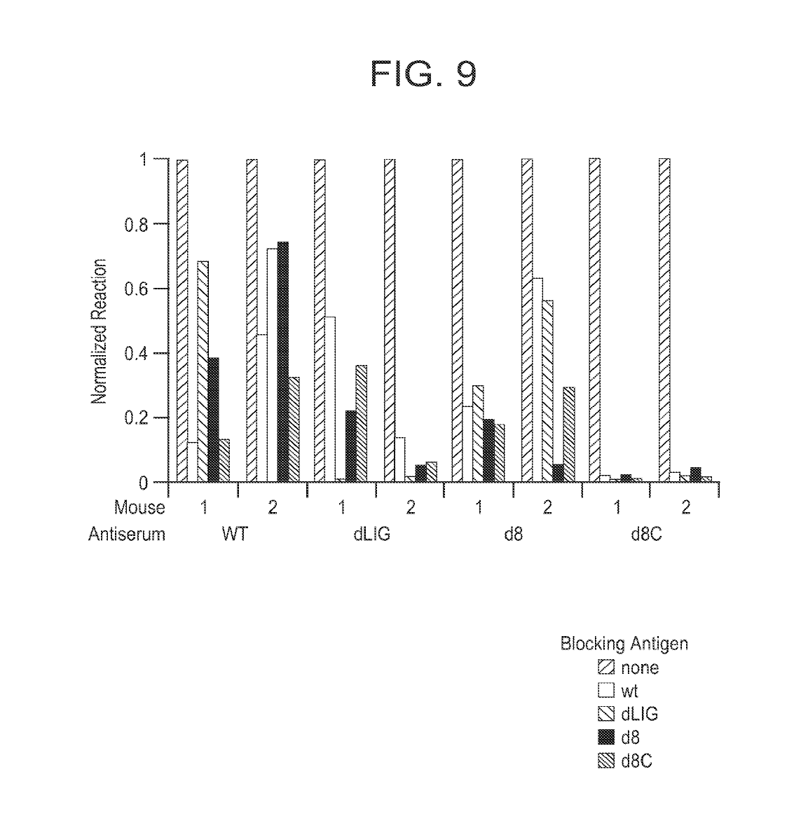

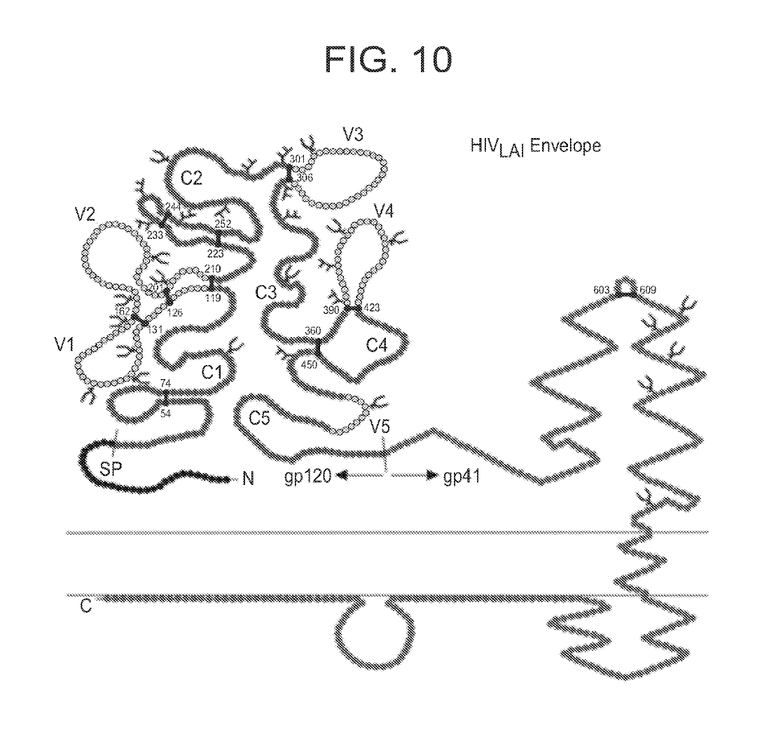

The location of the immunodominant antibody epitopes can be inferred from the patterns of cross-reactivity. Cross-reactivity of sera from two mice immunized with T4Hsp10 or a deletion variant was analyzed by competitive ELISA (FIG. 9). Each serum was incubated with a four-fold excess of antigen, relative to the expected IgG concentration, prior to incubation with the immobilized immunogen. Reactions for blocked sera were normalized to the reaction of the same serum incubated without blocking antigen. As expected, the immunogen blocked each serum with equal or greater effectiveness than any other antigen. However, there were clear differences in the extent to which the sera cross-reacted. Whereas T4Hsp10 blocked greater than 90% of the antibody raised against T4Hsp10, the deletion variants blocked an average of only 50%. In contrast, all four proteins, including T4Hsp10, blocked greater than 95% of the antibody raised against T4Hsp10d8C. Sera against T4Hsp10dLIG and T4Hsp10d8 exhibited intermediate levels of blocking by cross-reacting antigens. The exceptional cross-reactivity of T4Hsp10d8C antisera indicates a shift of immunodominance from the mobile loop to other epitopes that are shared by all four proteins. Reactivity with peptides corresponding to the mobile loop and other disordered segments was detected in the wild-type antisera. The more effective blocking of deletion-variant antisera was not due to low antibody responses against these immunogens. Antibody titers against the deletion variants were 6-10 times higher than the titer against T4Hsp10.

### Example 17

### Conclusions

The results with HIV gp120 and T4Hsp10 are remarkable in that, for both proteins, the structural modifications reduced T cell responses and increased antibody responses. Moreover, for both proteins, certain modifications increased the breadth of antibody specificity. At least some of the newly recruited epitopes were conformational, for example, as indicated by the increased CD4 blocking by gp120dss378 antisera and increased crossreactivity of the T4Hsp10d8C antisera. Thus, we propose that at least part of the shift in the immune responses is due to altered pathways of antigen processing, which leads to priming of different T cells and helping of different B cells. These data support a strategy for the design of a vaccine based on the HIV envelope glycoprotein.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All publications and patent applications, including U.S. application Ser. No. 61/103,793, mentioned in this specification are herein incorporated by reference to the same extent as if each independent publication or patent application was specifically and individually indicated to be incorporated by reference in their entirety.

What is claimed is:

1. A method of improving the immunogenicity of a polypeptide antigen, said method comprising removing or disrupting at least one intrachain disulfide bond of said antigen.

2. The method of claim 1, wherein said antigen can elicit a neutralizing antibody when administered to a mammal.

3. The method of claim 2, wherein said mammal is a human.

4. The method of claim 1, wherein said antigen is not completely denatured.

5. The method of claim 1, wherein said antigen is glycosylated.

6. The method of claim 1, wherein one or more cysteine residues in said antigen are substituted or deleted in order to remove or disrupt said at least one disulfide bond.

7. The method of claim 5, wherein said cysteine residues are substituted with alanine.

8. The method of claim 1, wherein said disulfide bond is removed or disrupted following exposure to a reducing agent.

9. The method of claim 1, wherein said antigen is a viral, fungal, bacterial, or parasitic antigen.

10. The method of claim 9, wherein said viral, fungal, bacterial, or parasitic antigen comprises a neutralizable epitope.

11. The method of claim 9, wherein said viral antigen is the human immunodeficiency virus (HIV) envelope glycoprotein, or fragment thereof.

12. The method of claim 11, wherein said HIV envelope glycoprotein is gp120.

13. The method of claim 11, wherein said HIV envelope glycoprotein is gp41.

14. The method of claim 11, wherein said HIV envelope glycoprotein is gp160.

15. The method of claim 11, wherein said intrachain disulfide bond forms a variable (V) loop region of the HIV envelope glycoprotein.

16. The method of claim 11, wherein said intrachain disulfide bond forms the variable loop (V3) region of the HIV envelope glycoprotein.

17. The method of claim 11, wherein said intrachain disulfide bond forms the variable loop (V4) region of the HIV envelope glycoprotein.

18. The method of claim 9, wherein said viral antigen is not derived from the hepatitis C virus (HCV).

19. A vaccine comprising a polypeptide antigen, wherein at least one intrachain disulfide bond in said polypeptide is removed or disrupted.

20. The vaccine of claim 19, wherein said antigen can elicit a neutralizing antibody when administered to a mammal.

21. The vaccine of claim 20, wherein said mammal is a human

22. The vaccine of claim 19, wherein said antigen is not completely denatured.

23. The vaccine of claim 19, wherein said antigen is glycosylated.

24. The vaccine of claim 19, wherein one or more cysteine residues in said antigen are substituted or deleted in order to remove or disrupt said at least one disulfide bond.

25. The vaccine of claim 24, wherein said cysteine residues are substituted with alanine.

26. The vaccine of claim 24, wherein said disulfide bond is removed or disrupted following exposure to a reducing agent.

27. The vaccine of claim 19, wherein said antigen is a viral, fungal, bacterial, or parasitic antigen.

28. The vaccine of claim 27, wherein said viral, fungal, bacterial, or parasitic antigen comprises a neutralizable epitope.

29. The vaccine of claim 27, wherein said viral antigen is the human immunodeficiency virus (HIV) envelope glycoprotein, or fragment thereof.

30. The vaccine of claim 29, wherein said HIV envelope glycoprotein is gp120.

31. The vaccine of claim 29, wherein said HIV envelope glycoprotein is gp41.

32. The vaccine of claim 29, wherein said HIV envelope glycoprotein is gp160.

33. The vaccine of claim 29, wherein said intrachain disulfide bond forms a variable (V) loop region of the HIV envelope glycoprotein.

34. The vaccine of claim 29, wherein said intrachain disulfide bond forms the variable loop (V3) region of the HIV envelope glycoprotein.

35. The vaccine of claim 29, wherein said intrachain disulfide bond forms the variable loop (V4) region of the HIV envelope glycoprotein.

36. The vaccine of claim 19, wherein said viral antigen is not derived from the hepatitis C virus (HCV).

37. An expression vector comprising a nucleic acid that encodes the sequence of the vaccine of claim 19.

38. A method of treating a mammal infected with or at risk of becoming infected with a pathogen comprising administering to said mammal the vaccine of claim 19.

39. The method of claim 38, wherein said mammal is a human.

40. A method of treating a mammal infected with or at risk of becoming infected with a pathogen comprising administering to said mammal the expression vector of claim 37.

41. The method of claim 40, wherein said mammal is a human.

42. A method of manufacturing a vaccine with enhanced immunogenicity in a mammal, said method comprising synthesizing the vaccine of claim 19.

43. A method of manufacturing a vaccine with enhanced immunogenicity in a mammal, said method comprising the steps of:
a) contacting the expression vector of claim 37 with a cell; and
b) isolating said polypeptide antigen.

44. The method of claim 42 or 43, wherein said antigen can elicit a neutralizing antibody when administered to a mammal.

45. The method of claim 42 or 43, wherein said mammal is a human.

46. The method of claim 42 or 43, wherein said antigen is not completely denatured.

47. The method of claim 42 or 43, wherein said antigen is glycosylated.

48. The method of claim 42 or 43, wherein one or more cysteine residues in said antigen are substituted or deleted in order to remove or disrupt said at least one disulfide bond.

49. The method of claim 48, wherein said cysteine residues are substituted with alanine.

50. The method of claim 42 or 43, wherein said disulfide bond is removed or disrupted following exposure to a reducing agent.

51. The method of claim 42 or 43, wherein said antigen is a viral, fungal, bacterial, or parasitic antigen.

52. The method of claim 51, wherein said viral, fungal, bacterial, or parasitic antigen comprises a neutralizable epitope.

53. The method of claim 51, wherein said viral antigen is the human immunodeficiency virus (HIV) envelope glycoprotein, or fragment thereof.

54. The method of claim 53, wherein said HIV envelope glycoprotein is gp120.

55. The method of claim 53, wherein said HIV envelope glycoprotein is gp41.

56. The method of claim 53, wherein said ITV envelope glycoprotein is gp160.

57. The method of claim 53, wherein said intrachain disulfide bond forms a variable (V) loop region of the HIV envelope glycoprotein.

58. The method of claim 53, wherein said intrachain disulfide bond forms the variable loop (V3) region of the HIV envelope glycoprotein.

59. The method of claim 53, wherein said intrachain disulfide bond forms the variable loop (V4) region of the HIV envelope glycoprotein.

60. The method of claim 51, wherein said viral antigen is not derived from the hepatitis C virus (HCV).

61. The method of claim 43, wherein said cell is a bacterial, fungal, insect, or animal cell.

62. The method of claim 61, wherein said animal cell is a mammalian cell.

63. A method of identifying an antibody that specifically binds a pathogenic antigen, said method comprising the steps of:
a) contacting an antibody with a viral, fungal, bacterial, or parasitic antigen, wherein at least one disulfide bond in said antigen has been removed or disrupted; and
b) identifying an antibody that binds said antigen with a dissociation constant of less than 10⁻⁶M.

64. The method of claim 63, wherein said antibody is identified from an antibody library.

65. The method of claim 63, wherein said antibody neutralizes said antigen.

66. The method of claim 63, wherein said viral, fungal, bacterial, or parasitic antigen comprises a neutralizable epitope.

67. The method of claim 63, wherein said pathogenic antigen is a human immunodeficiency virus (HIV) antigen.

68. The method of claim 66, wherein said ITV antigen is viral antigen is the HIV envelope glycoprotein, or fragment thereof.

69. The method of claim 68, wherein said HIV envelope glycoprotein is gp120.

70. The method of claim 68, wherein said HIV envelope glycoprotein is gp41.

71. The method of claim 68, wherein said HIV envelope glycoprotein is gp160.

72. The method of claim 68, wherein said intrachain disulfide bond forms a variable (V) loop region of the HIV envelope glycoprotein.

73. The method of claim 68, wherein said intrachain disulfide bond forms the variable loop (V3) region of the HIV envelope glycoprotein.

74. The method of claim 68, wherein said intrachain disulfide bond forms the variable loop (V4) region of the HIV envelope glycoprotein.

75. The method of claim 63, wherein said viral antigen is not derived from the hepatitis C virus (HCV).

76. A kit comprising:
a) the vaccine of claim 19;
b) a pharmaceutically acceptable carrier, excipient, or diluent; and
c) instructions for use thereof.

77. The kit of claim 76, further comprising an adjuvant.

78. A kit comprising:
a) the expression vector of claim 37;
b) a pharmaceutically acceptable carrier, excipient, or diluent; and
c) instructions for use thereof.

79. The kit of claim 78, further comprising an adjuvant.
